(19) United States
(12) Patent Application Publication
(10) Pub. No.: US 2026/0193316 A1
(43) Pub. Date: Jul. 9, 2026

(54) NOVEL CD20 PROTEIN

(71) Applicant: Malcorp Biodiscoveries Limited, Wellington (NZ)

(72) Inventors: Patricia Rubio-Reyes, Wellington (NZ); Rachel Perret, Wellington (NZ); Robert Weinkove, Wellington (NZ)

(21) Appl. No.: 19/133,133

(22) PCT Filed: Nov. 29, 2023
(86) PCT No.: PCT/NZ2023/050139

§ 371 Date: May 27, 2025

(30) Foreign Application Priority Data
Nov. 29, 2022 (NZ) 795049

(51) Int. Cl. C07K 14/705; C12N 15/86

(52) U.S. Cl. CPC C07K 14/70596; C12N 15/86; C12N 2740/15043

(57) ABSTRACT

The present invention is concerned with a human CD20 protein with modifications to one or more intracellular domains sufficient to reduce or abrogate intracellular signalling when attached to or associated with the membrane of the cell such as (e.g.) a T-cell, a natural killer cell, a B-cell, a myeloid cell, a pluripotent stem cell and a haematopoietic stem cell. The present invention further contemplates a nucleic acid encoding a modified human CD20 protein described herein, and to the utility of the modified human CD20 protein as a safety switch in chimeric antigen receptor T-cell therapy or as a selection marker for gene therapies.

### TECHNICAL FIELD

The present invention is concerned with a human CD20 protein which includes at least one mutation intended to reduce or abrogate intracellular signalling when attached to or associated with the membrane of a cell, including for example, T-cells, natural killer cells, B-cells, myeloid cells, haematopoietic stem cells, non-haematopoietic stem cells, pluripotent stem cells, and human cell lines. In particular, the present invention provides an engineered human CD20 protein comprising at least one mutation in at least one intracellular domain, including amino acid residues 1-56 or 210-297 as defined by SEQ ID NO: 1.

### BACKGROUND OF THE INVENTION

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated herein by reference in their entirety.

It is estimated that by 2025 the US Food and Drug Administration (FDA) will be approving between 10 and 20 gene therapies each year [1]. The rapid expansion of this field, targeting a broad range of diseases like blindness, immune and neuronal disorders, as well as cancers [2], highlights the urgent need for safety mechanisms to address potential short and long-term adverse effects (e.g. cell cytotoxicity in CAR T-cell therapy).

Adverse effects created by gene therapy may be regulated by incorporating a safety switch, such as a suicide gene, within the gene construct, or by including a cell elimination marker not normally present on the modified cells, allowing for antibody-mediated cytotoxicity [3]. Using antibodies already approved clinically, (e.g.) Cetuximab (anti-EGFR) [4] or Rituximab (anti-CD20) [5] offers an advantage for cell surface elimination markers over other types of regulation. The Federal Drug Agency (FDA) and European Medicines Agency (EMA) recommend that the cellular kinetics, bio-distribution and persistence of genetically-modified cells are assessed, and that long-term follow-up includes monitoring for late-onset events, including secondary malignancies, which the use of a single protein that can serve as both a safety switch and detection marker provides a solution for [6, 7].

The present invention seeks to address this clinical need by providing an engineered CD20 protein in which the intracellular domains have been modified to abrogate CD20 mediated signalling while retaining detection and killing by anti-CD20 antibodies including, for example, Rituximab, Obinutuzumab and Ocrelizumab

### SUMMARY OF THE INVENTION

The inventions described and claimed herein have many attributes and embodiments including, but not limited to, those set forth or described or referenced in this Summary of the Invention. It is not intended to be all inclusive and the inventions described and claimed herein are not limited to or by the features or embodiments identified in this Summary of the Invention, which is included for purposes of illustration only and not restriction.

In an aspect the present invention provides a modified human CD20 protein comprising:
(i) at least one mutation causing a truncation to any one or more of amino acid residues 1-56 set forth in SEQ ID NO: 1;
(ii) at least one mutation causing a truncation to any one or more of amino acid residues 210-297 set forth in SEQ ID NO: 1;
(iii) at least one mutation to a threonine or a serine located within amino acid residues 1-56 set forth in SEQ ID NO: 1;
(iv) at least one mutation to a threonine or a serine located within amino acid residues 210-297 set forth in SEQ ID NO: 1; or
(v) a combination comprising any one of (i) to (iv)

wherein the at least one mutation defined by any one of (i) to (v) causes reduced or abrogated intracellular signalling when the modified CD20 protein is attached to or associated with the membrane of a cell.

In another aspect the present invention provides a modified human CD20 protein comprising:
(i) at least one mutation causing a truncation to any one or more of amino acid residues 1-56 set forth in SEQ ID NO: 1; and
(ii) at least one mutation to a threonine or a serine located within amino acid residues 210-297 set forth in SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) at least one mutation to a threonine or a serine located within amino acid residues 1-56 set forth in SEQ ID NO: 1; and
(ii) at least one mutation causing a truncation to any one or more of amino acid residues 210-297 set forth in SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) at least one mutation to a threonine or a serine located within amino acid residues 1-56 set forth in SEQ ID NO: 1; and
(ii) at least one mutation to a threonine or a serine located within amino acid residues 210-297 set forth in SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) truncation of amino acid residues 1-50 set forth in SEQ ID NO: 1; and
(ii) at least one mutation to a threonine or a serine located within amino acid residues 210-297 set forth in SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) truncation of amino acid residues 1-50 set forth in SEQ ID NO: 1; and
(ii) at least one mutation to S225, S231 and T239 set forth in SEQ ID NO: 1

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) truncation of amino acid residues 1-50 set forth in SEQ ID NO: 1; and
(ii) at least one mutation to S225, S231 and T239 of SEQ ID NO: 1 comprising one or more of S225A, S231A and T239A,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) at least one mutation to a threonine or a serine located within amino acid residues 1-56 of SEQ ID NO: 1; and
(ii) truncation of amino acid residues 253-297 of SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) at least one mutation to T2, T3, S7, T11, S35, S36 and T51 of SEQ ID NO: 1; and
(ii) truncation of amino acid residues 253-297 of SEQ ID NO: 1,

wherein the at least one mutation defined by (i) and (ii) causes reduced or abrogated intracellular signalling when the modified CD20 protein is attached to or associated with the membrane of a cell.

In another aspect the present invention provides a modified human CD20 protein comprising:
(i) at least one mutation to T2, T3, S7, T11, S35, S36 and T51 of SEQ ID NO: 1 comprising one or more of T2A, T3A, S7A, T11A, S35A, S36A and T51A; and
(ii) truncation of amino acid residues 253-297 of SEQ ID NO: 1,

wherein the at least one mutation defined by (i) and (ii) causes reduced or abrogated intracellular signalling when the modified CD20 protein is attached to or associated with the membrane of a cell.

In yet another aspect the present invention provides a modified human CD20 protein comprising the sequence set forth in SEQ ID NO: 2, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 2.

In yet another aspect the present invention provides a modified human CD20 protein comprising the sequence set forth in SEQ ID NO: 3, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 3.

In yet another aspect the present invention provides a modified human CD20 protein comprising the sequence set forth in SEQ ID NO: 4, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 4.

In yet another aspect the present invention provides a modified human CD20 protein comprising the sequence set forth in SEQ ID NO: 5, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 5.

In yet another aspect the present invention provides a modified human CD20 protein comprising the sequence set forth in SEQ ID NO: 6, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 6.

In yet another aspect the present invention provides a modified human CD20 protein comprising the sequence set forth in SEQ ID NO: 7, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 7.

In yet another aspect the present invention provides a modified human CD20 protein comprising:
(i) at least one mutation causing a truncation to any one of amino acid residues 1-56 of SEQ ID NO: 1;
(ii) at least one mutation causing a truncation to any one of amino acid residues 210-297 of SEQ ID NO: 1;
(iii) at least one mutation to a threonine or a serine located within amino acid residues 1-56 of SEQ ID NO: 1;
(iv) at least one mutation to a threonine or a serine located within amino acid residues 210-297 of SEQ ID NO: 1; or
(v) a combination comprising any one of (i) to (iv); and
(vi) at least one mutation to any one of amino acid residues 142-188 of SEQ ID NO: 1 which eliminates binding by the monoclonal antibody Obinutuzumab

wherein any one of (i) to (v) causes reduced or abrogated intracellular signalling when the modified CD20 protein is attached to or associated with the membrane of a cell.

In yet another aspect the present invention provides a modified human CD20 protein comprising:
(i) at least one mutation causing a truncation to any one of amino acid residues 1-56 of SEQ ID NO: 1;
(ii) at least one mutation causing a truncation to any one of amino acid residues 210-297 of SEQ ID NO: 1;
(iii) at least one mutation to a threonine or a serine located within amino acid residues 1-56 of SEQ ID NO: 1;
(iv) at least one mutation to a threonine or a serine located within amino acid residues 210-297 of SEQ ID NO: 1; or
(v) a combination comprising any one of (i) to (iv); and
(vi) at least one mutation to any one of amino acid residues 142-188 of SEQ ID NO: 1 which eliminates binding by the monoclonal antibody Rituximab

wherein any one of (i) to (v) causes reduced or abrogated intracellular signalling when the modified CD20 protein is attached to or associated with the membrane of a cell.

In a further aspect the present invention provides a cell expressing any modified human CD20 protein as described herein.

In a further aspect the present invention provides a T-cell expressing any modified human CD20 protein as described herein.

In a further aspect the present invention provides a natural killer (NK) cell expressing any modified human CD20 protein as described herein.

In a further aspect the present invention provides a B-cell expressing any modified human CD20 protein as described herein.

In a further aspect the present invention provides a myeloid cell expressing any modified human CD20 protein as described herein.

In a further aspect the present invention provides a pluripotent cell expressing any modified human CD20 protein as described herein.

In a further aspect the present invention provides a haematopoietic stem cell expressing any modified human CD20 protein as described herein.

In a further aspect the present invention provides a haematopoietic stem cell line expressing any modified human CD20 protein as described herein.

In a further aspect the present invention provides a non-haematopoietic stem cell line expressing any modified human CD20 protein as described herein.

In a further aspect the present invention provides a human cell line expressing any modified human CD20 protein as described herein.

In yet a further aspect the present invention provides a nucleic acid molecule, such as a deoxyribose nucleic acid molecule (DNA), a messenger ribose nucleic acid molecule (mRNA) and a complementary deoxyribose nucleic acid molecule (cDNA), encoding any modified human CD20 protein described herein.

In yet another aspect the present invention provides a vector comprising a nucleic acid as described herein.

In yet another aspect the present invention provides an expression vector comprising a nucleic acid as described herein

### BRIEF DESCRIPTION OF THE FIGURES

FIG. 1 shows flow cytometry plots of HEK293 cells transfected with CD20-GFP and CD20 versions-GFP (diagonal shows that cells produce both GFP and CD20). Raji B cell line was used as a positive control for membrane detection of CD20.

FIG. 2 shows CD20's predicted phosphorylation sites. The residues represented in bold/underline are amino acids that are identical to mouse sequence and have a NetPhos score higher than 0.7; the residues in (round brackets) are amino acids identified as being identical to mouse sequence; the residues in [square brackets] are amino acids with a NetPhos score higher than 0.7; the residue in grey is an amino acid identified by the crystal structure; the residue in grey underline is an amino acid identified using all three methods, refer to Example 2.

FIG. 3 shows detection of surface CD20WT or modified CD20 on HEK293 cell line (upper panels) and primary T cells (lower panels) evaluated by labelling cells with Rituximab conjugated to AF405.

FIG. 4A shows total Rituximab-mediated cell death of CD20KO HG3 cells, either untransduced or transduced with CD20WT, C₁C₃ or C₁C₂₅₂.

FIG. 4B shows transduction efficiencies of CD20WT, C₁C₃ or C₁C₂₅₂, in CD20KO HG3 cells compared to untransduced cells.

FIG. 5 shows remaining GFP+ cells in mice blood (% of CD45.1 cells) after treatment with 2H7 antibody or an isotype control. Bars represent the mean % of 5 mice and error bars the SEM.

FIG. 6 shows detection of cell surface CD20 by Obinutuzumab, Rituximab or L27 antibodies in CD20KO HG3 cells transduced with CD20WT, C₁C₃ or C₁C₂₅₂ and untransduced control cells FIG. 7 shows Ca²⁺ influx induced by hyper-crosslinking of CD20WT, C₁C₃ and C₁C₂₅₂ in CD20KO HG3 cells compared to untransduced control evaluated by flow cytometry. Bars represent the MFI of 3 samples and error bars the SEM.

FIG. 8A shows overall phosphorylation status of CREB, WINK, GSK, ERK, STAT 5 and 6, Lyn, P53 and RSK upon CD20 activation by Rituximab binding measured by phospho-Western Blot.

FIG. 8B shows the difference in phosphorylation of kinases relevant for both B and T cell activation. Bars represent the mean difference and dots the individual measurements for 2 replicates per condition.

FIG. 9A shows identification of key binding residues in the CD20 epitope required by Rituximab and Obinutuzumab based on published information [8]. Asparagine (N) residues were individually substituted with alanine (A) to specifically abrogate binding of one or the other antibody.

FIG. 9B shows the detection of surface CD20 by Rituximab or Obinutuzumab in HEK293 cells transduced with CD20WT, ΔObinutuzumab mutant (N176A substitution to specifically abrogate Obinutuzumab binding) and ΔRituximab mutant (N171A substitution to specifically abrogate Rituximab binding

FIG. 10 shows amino acid sequences for exemplary modified human CD20 proteins according to the present invention. X=any naturally or non-naturally occurring amino acid residue.

### DETAILED DESCRIPTION

### General Definitions

Unless specifically defined otherwise, all technical and scientific terms used herein shall be taken to have the same meaning as commonly understood by one of ordinary skill in the art to which the inventions belong (for example, in immunology, immunohistochemistry, protein chemistry, and biochemistry).

Unless otherwise indicated, the recombinant protein and immunological techniques utilized in the present invention are standard procedures well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al., (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The term “a” or “an” refers to one or more than one of the entity specified; for example, “a receptor” or “a nucleic acid molecule” may refer to one or more receptor or nucleic acid molecule, or at least one receptor or nucleic acid molecule. As such, the terms “a” or “an”, “one or more” and “at least one” can be used interchangeably herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally equivalent products, compositions and methods are clearly within the scope of the invention, as described herein.

Any example or embodiment described herein shall be taken to apply mutatis mutandis to any other example or embodiment unless specifically stated otherwise.

### Selected Definitions

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide typically of sizes less than a total of 15-20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino terminus to a carboxy terminus.

The terms “amino acid” “amino acid residue,” “amino acid sequence,” or polypeptide sequence include naturally occurring amino acids (including L and D isosteriomers) and, unless otherwise limited, also include known analogues of natural amino acids that can function in a similar manner as naturally occurring amino acids, such as, e.g., selenocysteine, pyrrolysine, N-formylmethionine, gamma-carboxyglutamate, hydroxyprolinehypusine, pyroglutamic acid, and selenomethionine. The amino acids referred to herein are described by shorthand designations as follows in Table A:

TABLE A
Amino Acid Nomenclature

| Name | 3-Letter Code | 1-Letter Code |
|---|---|---|
| Alanine | Ala | A |
| Arginine | Arg | R |
| Asparagine | Asn | N |
| Aspartic Acid (Aspartate) | Asp | D |
| Cysteine | Cys | C |
| Glutamic Acid (Glutamate) | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | T |
| Tyrosine | Tyr | Y |
| Valine | Val | V |

The term “antibody” refers to an immunoglobulin molecule capable of selectively binding to a target, such as human CD20, by virtue of an antigen binding site contained within at least one variable region. This term includes four chain antibodies (e.g., two light chains and two heavy chains), recombinant or modified antibodies (e.g., chimeric antibodies, humanized antibodies, primatised antibodies, de-immunized antibodies, half antibodies, bispecific antibodies) and single domain antibodies such as domain antibodies and heavy chain only antibodies (e.g., camelid antibodies or cartilaginous fish immunoglobulin new antigen receptors (IgNARs)). An antibody generally comprises constant domains, which can be arranged into a constant region or constant fragment or fragment crystallisable (Fc). Preferred forms of antibodies comprise a four-chain structure as their basic unit. Full-length antibodies comprise two heavy chains (~50-70 kDa) covalently linked and two light chains (~23 kDa each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH which is ~330-440 amino acids in length). The light chain variable region is aligned with the variable region of the heavy chain. The antibody heavy chain can comprise 2 or more additional CH domains (such as, CH2, CH3 and the like) and can comprise a hinge region can be identified between the CH1 and Cm constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (preferably human) antibody. The term “antibody” encompasses not only intact polyclonal or monoclonal antibodies, but also variants, fusion proteins comprising an antibody portion with an antigen binding site, humanised antibodies, human antibodies, chimeric antibodies, primatised antibodies, de-immunised antibodies or veneered antibodies.

The term “conservative substitution” with regard to a polypeptide, refers to a change in the amino acid composition of the polypeptide that does not substantially alter the function and structure of the overall polypeptide (see Creighton, Proteins: Structures and Molecular Properties (W. H. Freeman and Company, New York (2nd ed., 1992)).

As used herein, the term “expressed,” “expressing” or “expresses” refers to transcription and/or translation of a polynucleotide or nucleic acid into a polypeptide or protein. The produced polypeptides or proteins may remain intracellular, become a component of the cell surface membrane or be secreted into an extracellular space.

As used herein, cells which express a significant amount of CD20 on at least one cellular surface are “CD20 positive cells” or “CD20⁺ cells” and are cells physically coupled to significant amounts of the extracellular target biomolecule CD20.

The term “encode” as used herein refers to the inherent property of a specific sequence of nucleotides in a polynucleotide, such as a gene, cDNA, or mRNA, as a template in biological processes for the synthesis of other polymers and macromolecules having defined nucleotide sequences (e.g., rRNA, tRNA, and mRNA) or defined amino acid sequences and biological properties derived therefrom. Thus, a gene, cDNA, or RNA encodes a protein if transcription and translation of mRNA corresponding to the gene produces the protein in a cell or other biological system. Both the coding strand, the nucleotide sequence of which is identical to the mRNA sequence and is typically provided in the sequence listing, and the non-coding strand, which serves as a template for transcription of a gene or cDNA, may be referred to as encoding the protein or other product of the gene or cDNA.

As used herein, the term “epitope” refers to the portion of an antigen (e.g., human CD20) that specifically interacts with an antibody molecule. These moieties, referred to herein as epitope determinants, typically comprise or are part of an element such as an amino acid side chain or a sugar side chain. Epitope determinants can be defined, for example, by methods known in the art or disclosed herein, e.g., by crystallography or by hydrogen deuterium exchange. At least one or some of the portions of the antibody molecule that specifically interact with the epitope determinants are typically located in the CDRs. Typically, epitopes have specific three-dimensional structural features. Typically, epitopes have a specific charge profile. Some epitopes are linear epitopes, while others are conformational epitopes. An exemplary epitope according to the present invention is that defined by amino acid residues 167 to 183 of SEQ ID NO: 1, namely CEPANPSEKNSPSTQYC (SEQ ID NO: 13) which incorporates amino acid residues critical for binding of the clinically approved monoclonal antibodies Rituximab and Obinutuzumab.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein the term “endogenous” refers to any material from or produced inside an organism, cell, tissue or system.

As used herein the term “exogenous” refers to any material introduced from or produced outside an organism, cell, tissue or system.

As used herein the term “expression” is defined as the transcription and/or translation of a particular nucleotide sequence driven by its promoter.

The term “expression vector” refers to a vector comprising a recombinant polynucleotide comprising an expression control sequence operably linked to a nucleotide sequence to be expressed. The expression vector contains sufficient cis-acting elements for expression; other elements for expression may be provided by the host cell or in an in vitro expression system. Expression vectors include all those known in the art, including cosmids, plasmids (e.g., naked or contained in liposomes), and viruses (e.g., lentiviruses, retroviruses, adenoviruses, and adeno-associated viruses) that incorporate the recombinant polynucleotide.

The term “identity” refers to subunit sequence identity between two polymeric molecules, for example between two nucleic acid molecules, such as between two DNA molecules or two RNA molecules, or between two polypeptide molecules. When a subunit site in both molecules is occupied by the same monomeric subunit; for example, if a position in each of two polypeptide or protein molecules is occupied by a serine, they are homologous or identical at that position. Homology between two sequences is a direct function of the number of matching or homologous positions; for example, two sequences are 50% homologous if half the sites (e.g., five positions in a polymer ten subunits in length) in the two sequences are the same; if 90% of the sites (e.g., 9 out of 10) are matched or homologous, then the two sequences are 90% the same.

The term “intracellular signalling domain” as used herein refers to the intracellular portion of a molecule, for example, CD20. The intracellular signalling domain(s) generate a signal that promotes (e.g.) an immune effector function of a CAR-containing cell, such as a CAR T-cell. Examples of immune effector functions, such as in CAR T-cells, include cytolytic and helper activities, including secretion of cytokines. In certain examples, the intracellular signalling domain is a portion of a protein that transduces effector function signals and directs the cell to perform specialized functions. Although the entire intracellular signalling domain may be used, in many cases, the entire chain need not be used. To the extent that truncated portions of intracellular signalling domains are used, such truncated portions may be used in place of the entire chain, so long as they transduce effector function signals. The term intracellular signalling domain is therefore intended to include any truncated portion of an intracellular signalling domain sufficient to transduce an effector function signal.

The term “isolated” as applied to the protein or polypeptide sequences disclosed herein is used to refer to sequences that are removed from their natural cellular or other naturally-occurring biological environments. An isolated molecule may be obtained by any method or combination of methods including biochemical, recombinant, and synthetic techniques. The polypeptide sequences may be prepared by at least one purification step.

The term “lentivirus” refers to a species of the retrovirus family. Lentiviruses are unique among retroviruses in that they are capable of infecting non-dividing cells; they can deliver large amounts of genetic information into the DNA of host cells, and thus they are one of the most efficient methods of gene delivery vectors. HIV, SIV and FIV are examples of lentiviruses.

The term “lentiviral vector” refers to a vector derived from at least a portion of the lentiviral genome, and specifically includes self-inactivating lentiviral vectors such as those provided in [44]. Other examples of lentiviral vectors that can be used in the clinic include, but are not limited to, for example, those from Oxford BioMedicaGene delivery technology or LentigenVector systems. Non-clinical varieties of lentiviral vectors are also available and will be known to those skilled in the art.

The terms “nucleic acid” or “polynucleotide” refers to deoxyribonucleic acid (DNA) or ribonucleic acid (RNA) in either single- or double-stranded form, and polymers thereof. The term “nucleic acid” includes a gene, cDNA or mRNA. In one example, the nucleic acid molecule is synthetic (e.g., chemically synthesized) or recombinant. Unless specifically limited, the term includes nucleic acids containing analogs or derivatives of natural nucleotides that have similar binding properties as the reference nucleic acid and are metabolized in a manner similar to naturally occurring nucleotides. Unless otherwise indicated, a particular nucleic acid sequence also implicitly includes conservatively modified variants thereof (e.g., degenerate codon substitutions), alleles, orthologs, SNPs, and complementary sequences, as well as the sequence explicitly indicated. In particular, degenerate codon substitutions may be achieved by generating sequences in which three positions of one or more selected (or all) codons are substituted with mixed base and/or deoxyinosine residues.

The terms “nucleic acid encoding an amino acid sequence” or “nucleic acid encoding a protein” includes all nucleotide sequences that are degenerate versions of each other and that encode the same amino acid sequence or protein. In certain examples, the nucleic acid encoding the polypeptide or protein may contain an intron(s).

The terms “peptide”, “polypeptide” and “protein” are used interchangeably and refer to a compound consisting of amino acid residues covalently linked by peptide bonds. The protein or peptide must contain at least two amino acids, and there is no limit to the maximum number of amino acids that can comprise the sequence of the protein or peptide. Polypeptides include any peptide or protein comprising two or more amino acids joined to each other by peptide bonds. As used herein, the term refers to both short chains (which are also commonly referred to in the art as, for example, peptides, oligopeptides, and oligomers) and longer chains (which are also commonly referred to in the art as proteins, of which there are various types). “Polypeptide” includes, for example, biologically active fragments, substantially homologous polypeptides, oligopeptides, homodimers, heterodimers, variants of polypeptides, modified polypeptides, derivatives, analogs, fusion proteins, and the like. The polypeptide includes a native peptide, a recombinant peptide, or a combination thereof.

The term “polynucleotide” as used herein is defined as a chain of nucleotides. Furthermore, nucleic acids are polymers of nucleotides. Thus, the terms “nucleic acid” and “polynucleotide” may be used interchangeably in this specification. A person skilled in the art would understand from his/her common general knowledge that nucleic acids are polynucleotides, which can be hydrolyzed into monomeric “nucleotides.” The monomeric nucleotides can be hydrolysed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “promoter” refers to a DNA sequence recognized by, or introduced into, the synthetic machinery of a cell, which is required to initiate specific transcription of a polynucleotide sequence.

The term “promoter/regulatory sequence” refers to a nucleic acid sequence required for expression of a gene product operably linked to a promoter/regulatory sequence. In some cases, the sequence may be a core promoter sequence, and in other cases, the sequence may also include enhancer sequences and other regulatory elements required for expression of the gene product. The promoter/regulatory sequence may, for example, be a sequence which expresses the gene product in a tissue-specific manner.

The term “specifically binds” refers to an antibody or ligand that recognises and binds to a binding partner (e.g., a stimulatory tumour antigen) present in a sample, but which antibody or ligand does not substantially recognise or bind to other molecules in the sample.

In this specification, reference to “at least one mutation to a serine or threonine” may include reference to any one or more of (e.g.) T2, T3, S7, T11, S25, S35, S36, T41, S43, S49, T51, S221, S225, S231, T239, T250, T252, S253, S254, T275, T277, S288, S289, S295 and S296 of SEQ ID NO: 1 and includes a substitution mutation, a deletion mutation or an insertion mutation involving an amino acid residue that is equivalent to a threonine at position 2, a threonine at position 3, a serine at position 7, a threonine at position 11, a serine at position 25, a serine at position 35, a serine at position 36, a threonine at position 41, a serine at position 43, a serine at position 49, a threonine at position 51, a serine at position 221, a serine at position 225, a serine at position 231, a threonine at position 239, a threonine at position 250, a threonine at position 252, a serine at position 253, a serine at position 254, a threonine at position 275, a threonine at position 277, a serine at position 288, a serine at position 289, a serine at position 295 and a serine at position 296 defined by the reference human CD20 sequence set forth in SEQ ID NO: 1.

Term “variant” as used herein refers to protein or polypeptide sequences different from the specifically identified sequences, wherein (e.g.) one to several amino acid residues are deleted, substituted, or added. Variants may be naturally occurring allelic variants, or non-naturally occurring variants. Variants may be from the same or from other species and may encompass homologues, paralogues and orthologues. In certain embodiments, variants of the polypeptides useful in the invention have biological activities including signal peptide activity or antigenic-binding properties that are the same or similar to those of the parent polypeptides. The term “variant” with reference to polypeptides encompasses all forms of polypeptides as defined herein.

Variant polypeptide sequences exhibit at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to a sequence of the present invention. With regard to polypeptides, identity is found over a comparison window of at least 297 amino acid positions.

Polypeptide variants also encompass those which exhibit a similarity to one or more of the specifically identified sequences that is likely to preserve the functional equivalence of those sequences, including those which could not reasonably be expected to have occurred by random chance.

Polypeptide sequence identity and similarity can be determined in the following manner. The subject polypeptide sequence is compared to a candidate polypeptide sequence using BLASTp (from the BLAST suite of programs, version 2.2.18 [April 2008]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters: bl2seq-i peptideseq1-j peptideseq2-F F-p blastp. The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match. Variant polypeptide sequences commonly exhibit an E value of less than 1×10⁻⁵, less than 1×10⁻⁶, less than 1×10⁻⁹, less than 1×10⁻¹², less than 1×10⁻¹⁵, less than 1×10⁻¹⁸ or less than 1×10⁻²¹ when compared with any one of the specifically identified sequences. Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235) as discussed above are also suitable global sequence alignment programs for calculating polypeptide sequence identity. Use of BLASTp is preferred for use in the determination of polypeptide variants according to the present invention.

The term “vector” as used herein is a composition of matter which comprises an isolated nucleic acid and which can be used to deliver the isolated nucleic acid to a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term “vector” should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors etc.

The acronym “WT” or “W-T” as used herein is intended to mean “wild-type” which when used (e.g.) in the context of a human CD20 is intended to mean the composition of a CD20 protein which typically found in situ.

### DETAILED DESCRIPTION

Mutant (Human) CD20—Cytoplasmic Domain Modifications The physiological role, regulation and ligand of CD20 are unclear [9], but functional studies suggest that CD20 is required for efficient B-cell receptor signalling [10] and that it is directly involved in calcium entry, which proper function depends on association into lipid rafts [11].

CD20 is the target of clinically licensed monoclonal antibodies including Rituximab and Obinutuzumab, which are routinely used to deplete lymphoid cells in the treatment of B-cell cancers and autoimmune diseases and have a well-established safety profile.

Rapid elimination of gene-transduced or gene-transfected cells may be required in the event of significant on-target off-tissue toxicity, or off-target toxicity. However, the binding of Rituximab or Obinutuzumab could lead to transient activation of CD20-expressing cells, via calcium influx and initiation of intracellular signalling cascades (e.g.) phosphorylation of kinases [12], [60], [61]. This risks a paradoxical exacerbation of toxicities due to the gene-modified cells, which may be a particular risk among recipients with defective complement dependent cytotoxicity (CDC) or antibody-dependent cellular toxicity (ADCC) due to recent cytotoxic or immunosuppressant use, or in patients with an underlying immunosuppressive disorder.

The main mechanisms of anti-CD20 antibody-mediated cell killing are CDC and ADCC [13], although one proposed mechanism for therapeutic B cell apoptosis is direct cytotoxicity mediated via src-family kinases as a consequence of lipid raft clustering [14]. Given that both N- and C-termini are located intracellularly (i.e. as cytoplasmic domains), Applicants designed a series of CD20 truncations and chimeric proteins in an attempt to abrogate CD20 signalling but retain antibody binding, preserving antibody dependent apoptosis.

The initial strategy employed was to truncate large portions of the cytoplasmic domains and to combine the cytoplasmic and transmembrane domains of CD20. Significant modifications to the membrane spanning 4a (MS4a) protein may affect the protein structure and its ability to traffic to and be correctly associated with the cell membrane.

Applicants also combined CD20's minimal antibody binding epitope with different signal peptides, transmembrane and cytoplasmic domains known to promote good surface protein trafficking and expression on cell membranes [15]. Applicants initially tested human granulocyte-macrophage colony-stimulating factor receptor (GM-CSFR) signal peptide and epidermal growth factor receptor (EGFR) transmembrane domain, as truncating EGFR's intracellular domain leads to a membrane bound protein [15].

Next, Applicants combined different lengths of CD20's antibody binding region with CD28 signal peptide and with transmembrane and cytoplasmic domains derived from CD28 and contactin-associated protein-like 2 (CASPR2) to generate chimeric proteins. This is because constructs incorporating CD28 and CASPR2 transmembrane/cytoplasmic domains yield membrane bound proteins [16, 17].

The different sequence constructs developed for these initial experiments are presented in Table 1 in Example 2.

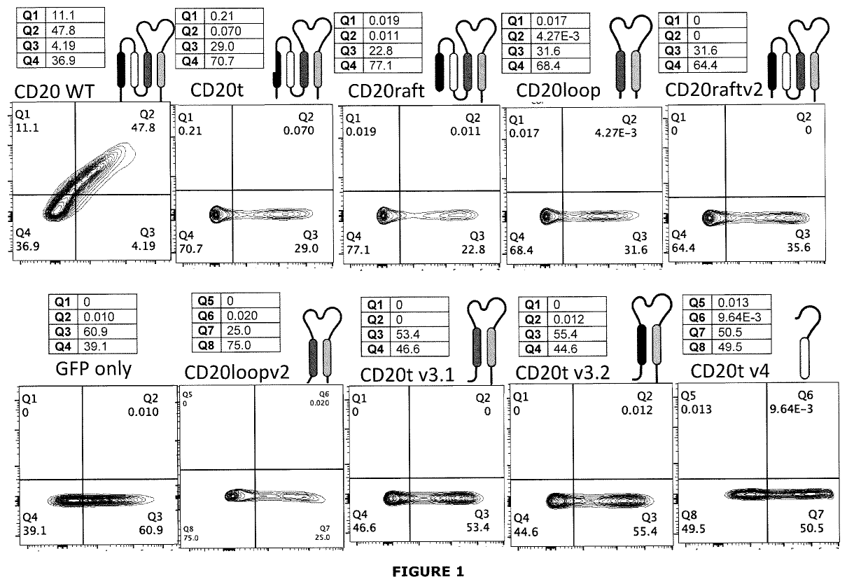

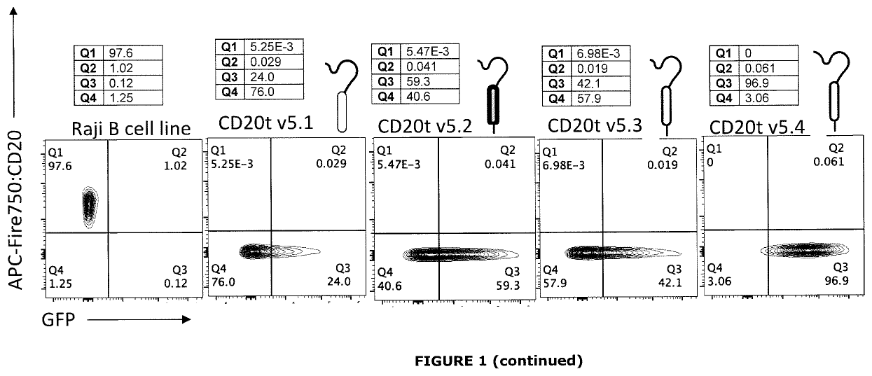

The initial results, which are captured in FIG. 1, were not expected. None of the constructs presented in Table 1 could be detected on the membrane of a HEK293 cell line. It is, however, important to note expression of the CD20-encoding transgene was detected in these cells as reflected by the production of a green florescent protein tag.

By way of illustration only, neither the CD20t v4 construct nor the CD20t v5.3 constructs, in which amino acids from the extracellular domain of CD20 were fused to different signal peptides (e.g. GM-CSFR and CD28) and transmembrane/cytoplasmic domains (e.g. EGFR and CASPR2) resulted in membrane expression in a HEK293 cell line. This is particularly surprising given previous reports in the literature which document successful transmembrane trafficking/expression for proteins incorporating these domains [4, 16, 17].

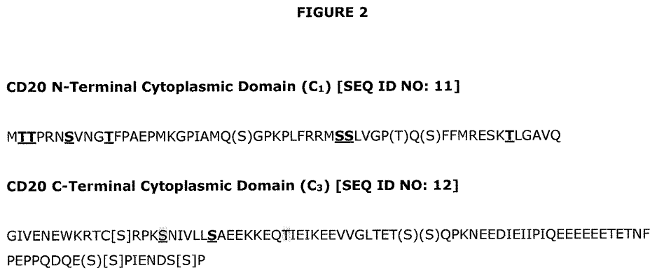

In response to these unexpected observations, Applicants modified their approach to the development of a membrane bound non-signalling CD20 molecule. Refer to the alternate strategy outlined in Examples 3-6, read in conjunction with FIGS. 2-5.

In brief, Applicant's alternate approach focused on targeting phosphorylated residues within CD20's cytoplasmic domains since CD20 is heavily phosphorylated on serine and threonine residues in normal and malignant B cells and this process is linked to B cell proliferation [18]. Rituximab binding to CD20 initiates a cascade of signals that may play a role in antibody-mediated cell cytotoxicity. CD20 is associated with lyn, fyn, lck and p75/85 kinase [19] and its engagement leads to the activation of PLCy via src-family kinases [20]. CD20's cytoplasmic sequences contain 15 serine residues, 11 threonine residues and no tyrosine residues. Refer to FIG. 2. CD20 therefore has 26 possible phosphorylation sites, although the literature only reports direct evidence of two [21].

Accordingly, in an aspect of the present invention there is provided a modified human CD20 protein comprising:
(i) at least one mutation causing a truncation to any one or more of amino acid residues 1-56 set forth in SEQ ID NO: 1;
(ii) at least one mutation causing a truncation to any one or more of amino acid residues 210-297 set forth in SEQ ID NO: 1;
(iii) at least one mutation to a threonine or a serine located within amino acid residues 1-56 set forth in SEQ ID NO: 1;
(iv) at least one mutation to a threonine or a serine located within amino acid residues 210-297 set forth in SEQ ID NO: 1; or
(v) a combination comprising any one of (i) to (iv)

wherein any one of (i) to (v) causes reduced or abrogated intracellular signalling when the modified CD20 protein is attached to, or associated with the membrane of a cell.

In an example according to this and all other aspects of the present invention, the at least one mutation associated with feature (i) causes truncation of any one of the following amino acid residue(s) forth in SEQ ID NO: 1: amino acid residue 1 defined by 5′-M-3′ (SEQ ID NO: 26), amino acid residues 1-2 defined by 5′-MT-3′ (SEQ ID NO: 27), amino acid residues 1-3 defined by 5′-MTT-3′ (SEQ ID NO: 28), amino acid residues 1-4 defined by 5′-MTTP-3′ (SEQ ID NO: 29), amino acid residues 1-5 defined by 5′-MTTPR-3′ (SEQ ID NO: 30), amino acid residues 1-6 defined by 5′-MTTPRN-3′ (SEQ ID NO: 31), amino acid residues 1-7 defined by 5′-MTTPRNS-3′ (SEQ ID NO: 32), amino acid residues 1-8 defined by 5′-MTTPRNSV-3′ (SEQ ID NO: 33), amino acid residues 1-9 defined by 5′-MTTPRNSVN-3′ (SEQ ID NO: 34), amino acid residues 1-10 defined by 5′-MTTPRNSVNG-3′ (SEQ ID NO: 35), amino acid residues 1-11 defined by 5′-MTTPRNSVNGT-3′ (SEQ ID NO: 36), amino acid residues 1-12 defined by 5′-MTTPRNSVNGTF-3′ (SEQ ID NO: 37), amino acid residues 1-13 defined by 5′-MTTPRNSVNGTFP-3′ (SEQ ID NO: 38), amino acid residues 1-14 defined by 5′-MTTPRNSVNGTFPA-3′ (SEQ ID NO: 39), amino acid residues 1-15 defined by 5′-MTTPRNSVNGTFPAE-3′ (SEQ ID NO: 40), amino acid residues 1-16 defined by 5′-MTTPRNSVNGTFPAEP-3′ (SEQ ID NO: 41), amino acid residues 1-17 defined by 5′-MTTPRNSVNGTFPAEPM-3′ (SEQ ID NO: 42), amino acid residues 1-18 defined by 5′-MTTPRNSVNGTFPAEPMK-3′ (SEQ ID NO: 43), amino acid residues 1-19 defined by 5′-MTTPRNSVNGTFPAEPMKG-3′ (SEQ ID NO: 44), amino acid residues 1-20 defined by 5′-MTTPRNSVNGTFPAEPMKGP-3′ (SEQ ID NO: 45), amino acid residues 1-12 defined by 5′-MTTPRNSVNGTFPAEPMKGPI-3′ (SEQ ID NO: 46), amino acid residues 1-22 defined by 5′-MTTPRNSVNGTFPAEPMKGPIA-3′ (SEQ ID NO: 47), amino acid residues 1-23 defined by 5′-MTTPRNSVNGTFPAEPMKGPIAM-3′ (SEQ ID NO: 48), amino acid residues 1-24 defined by 5′-MTTPRNSVNGTFPAEPMKGPIAMQ-3′ (SEQ ID NO: 49), amino acid residues 1-25 defined by 5′-MTTPRNSVNGTFPAEPMKGPIAMQS-3′ (SEQ ID NO: 50), amino acid residues 1-26 defined by 5′-MTTPRNSVNGTFPAEPMKGPIAMQSG-3′ (SEQ ID NO: 51), amino acid residues 1-27 defined by 5′-MTTPRNSVNGTFPAEPMKGPIAMQSGP-3′ (SEQ ID NO: 52), amino acid residues 1-28 defined by 5′-MTTPRNSVNGTFPAEPMKGPIAMQSGPK-3′ (SEQ ID NO: 53), amino acid residues 1-29 defined by 5′-MTTPRNSVNGTFPAEPMKGPIAMQSGPKP-3′ (SEQ ID NO: 54), amino acid residues 1-30 defined by 5′-MTTPRNSVNGTFPAEPMKGPIAMQSGPKPL-3′ (SEQ ID NO: 55), amino acid residues 1-31 defined by 5′-MTTPRNSVNGTFPAEPMKGPIAMQSGPKPLF-3′ (SEQ ID NO: 56), amino acid residues 1-32 defined by 5′-MTTPRNSVNGTFPAEPMKGPIAMQSGPKPLFR-3′ (SEQ ID NO: 57), amino acid residues 1-33 defined by 5′-MTTPRNSVNGTFPAEPMKGPIAMQSGPKPLFRR-3′ (SEQ ID NO: 58), amino acid residues 1-34 defined by 5′-MTTPRNSVNGTFPAEPMKGPIAMQSGPKPLFRRM-3′ (SEQ ID NO: 59), amino acid residues 1-35 defined by 5′-MTTPRNSVNGTFPAEPMKGPIAMQSGPKPLFRRMS-3′ (SEQ ID NO: 60), amino acid residues 1-36 defined by 5′-MTTPRNSVNGTFPAEPMKGPIAMQSGPKPLFRRMSS-3′ (SEQ ID NO: 61), amino acid residues 1-37 defined by 5′-MTTPRNSVNGTFPAEPMKGPIAMQSGPKPLFRRMSSL-3′ (SEQ ID NO: 62), amino acid residues 1-38 defined by 5′-MTTPRNSVNGTFPAEPMKGPIAMQSGPKPLFRRMSSLV-3′ (SEQ ID NO: 63), amino acid residues 1-39 defined by 5′-MTTPRNSVNGTFPAEPMKGPIAMQSGPKPLFRRMSSLVG-3′ (SEQ ID NO: 64), amino acid residues 1-40 defined by 5′-MTTPRNSVNGTFPAEPMKGPIAMQSGPKPLFRRMSSLVGP-3′ (SEQ ID NO: 65), amino acid residues 1-41 defined by 5′-MTTPRNSVNGTFPAEPMKGPIAMQSGPKPLFRRMSSLVGPT-3′ (SEQ ID NO: 66), amino acid residues 1-42 defined by 5′-MTTPRNSVNGTFPAEPMKGPIAMQSGPKPLFRRMSSLVGPTQ-3′ (SEQ ID NO: 67), amino acid residues 1-43 defined by 5′-MTTPRNSVNGTFPAEPMKGPIAMQSGPKPLFRRMSSLVGPTQS-3′ (SEQ ID NO: 68), amino acid residues 1-44 defined by 5′-MTTPRNSVNGTFPAEPMKGPIAMQSGPKPLFRRMSSLVGPTQSF-3′ (SEQ ID NO: 69), amino acid residues 1-45 defined by 5′-MTTPRNSVNGTFPAEPMKGPIAMQSGPKPLFRRMSSLVGPTQSFF-3′ (SEQ ID NO: 70), amino acid residues 1-46 defined by 5′-MTTPRNSVNGTFPAEPMKGPIAMQSGPKPLFRRMSSLVGPTQSFFM-3′ (SEQ ID NO: 71), amino acid residues 1-47 defined by 5′-MTTPRNSVNGTFPAEPMKGPIAMQSGPKPLFRRMSSLVGPTQSFFMR-3′ (SEQ ID NO: 72), amino acid residues 1-48 defined by 5′-MTTPRNSVNGTFPAEPMKGPIAMQSGPKPLFRRMSSL VGPTQSF FMRE-3′ (SEQ ID NO: 73), amino acid residues 1-49 defined by 5′-MTTPRNSVNGTFPAEPMKGPIAMQSGPKPLFRRMSSLVGPTQSFFMRES-3′ (SEQ ID NO: 74), amino acid residues 1-50 defined by 5′-MTTPRNSVNGTFPAEPM KGPIAMQSGPKPLFRRMSSLVG PTQSFFMRESK-3′ (SEQ ID NO: 75), amino acid residues 1-51 defined by 5′-MTTPRNSVNGTFPAEPMKGPIAMQSGPKPLFRRMSSLVGPTQSFFMRESKT-3′ (SEQ ID NO: 76), amino acid residues 1-52 defined by 5′-MTTPRNSVNGTFPAEPMKGPIAMQSGPKPLFRRMSSLVG PTQSFFMRESKTL-3′ (SEQ ID NO: 77), amino acid residues 1-53 defined by 5′-MTTPRNSVNGTFPAEPMKGPIAMQSGPKPLFRRMSSLVGPTQSFFMRESKTLG-3′ (SEQ ID NO: 78), amino acid residues 1-54 defined by 5′-MTTPRNSVNGTFPAEPMKGPIAMQSGPKPLFRRMSSLVG PTQSFFMRESKTLGA-3′ (SEQ ID NO: 79), amino acid residues 1-55 defined by 5′-MTTPRNSVNGTFPAEPMKGPIAMQSGPKPLFRRMSSLVGPTQSFFMRESKTLGAV-3′ (SEQ ID NO: 80), amino acid residues 1-56 defined by 5′-MTTPRNSVNGTFPAEPMKGPIAMQSGPKPLFR RMSSLVGPTQSFFMRESKTLGAVQ-3′ (SEQ ID NO: 81).

In another example according to this and other aspects of the present invention, the at least one mutation associated with feature (i) causes truncation of amino acid residues 1-50 set forth in SEQ ID NO: 1, namely 5′-MTTPRNSVNGTFPAEPMKGPIAMQSGPKPLFRRMSSLVG PTQSFFMRESK-3′ (SEQ ID NO: 75).

In an example according to this and other aspects of the present invention, the at least one mutation associated with feature (i) causes truncation of any one of the following amino acid residue(s) forth in SEQ ID NO: 1: amino acid residue 297 defined by 5′-P-3′ (SEQ ID NO: 82), amino acid residues 296-297 defined by 5′-SP-3′ (SEQ ID NO: 83), amino acid residues 295-297 defined by 5′-SSP-3′ (SEQ ID NO: 84), amino acid residues 294-297 defined by 5′-DSSP-3′ (SEQ ID NO: 85), amino acid residues 293-297 defined by 5′-NDSSP-3′ (SEQ ID NO: 86), amino acid residues 292-297 defined by 5′-ENDSSP-3′ (SEQ ID NO: 87), amino acid residues 291-297 defined by 5′-IENDSSP-3′ (SEQ ID NO: 88), amino acid residues 290-297 defined by 5′-PIENDSSP-3′ (SEQ ID NO: 89), amino acid residues 289-297 defined by 5′-SPIENDSSP-3′ (SEQ ID NO: 90), amino acid residues 288-297 defined by 5′-SSPIENDSSP-3′ (SEQ ID NO: 91), amino acid residues 287-297 defined by 5′-ESSPIENDSSP-3′ (SEQ ID NO: 92), amino acid residues 286-297 defined by 5′-QESSPIENDSSP-3′ (SEQ ID NO: 93), amino acid residues 285-297 defined by 5′-DQESSPIENDSSP-3′ (SEQ ID NO: 94), amino acid residues 284-297 defined by 5′-QDQESSPIENDSSP-3′ (SEQ ID NO: 95), amino acid residues 283-297 defined by 5′-PQDQESSPIENDSSP-3′ (SEQ ID NO: 96), amino acid residues 282-297 defined by 5′-PPQDQESSPIENDSSP-3′ (SEQ ID NO: 97), amino acid residues 281-297 defined by 5′-EPPQDQESSPIENDSSP-3′ (SEQ ID NO: 98), amino acid residues 280-297 defined by 5′-PEPPQDQESSPIENDSSP-3′ (SEQ ID NO: 99), amino acid residues 279-297 defined by 5′-FPEPPQDQESSPIENDSSP-3′ (SEQ ID NO: 100), amino acid residues 278-297 defined by 5′-NFPEPPQDQESSPIENDSSP-3′ (SEQ ID NO: 101), amino acid residues 277-297 defined by 5′-TNFPEPPQDQESSPIENDSSP-3′ (SEQ ID NO: 102), amino acid residues 276-297 defined by 5′-ETNFPEPPQDQESSPIENDSSP-3′ (SEQ ID NO: 103), amino acid residues 275-297 defined by 5′-TETNFPEPPQDQESSPIENDSSP-3′ (SEQ ID NO: 104), amino acid residues 274-297 defined by 5′-ETETNFPEPPQDQESSPIENDSSP-3′ (SEQ ID NO: 105), amino acid residues 273-297 defined by 5′-EETETNFPEPPQDQESSPIENDSSP-3′ (SEQ ID NO: 106), amino acid residues 272-297 defined by 5′-EEETETNFPEPPQDQESSPIENDSSP-3′ (SEQ ID NO: 107), amino acid residues 271-297 defined by 5′-EEEETETNFPEPPQDQESSPIENDSSP-3′ (SEQ ID NO: 108), amino acid residues 270-297 defined by 5′-EEEEETETNFPEPPQDQESSPIENDSSP-3′ (SEQ ID NO: 109), amino acid residues 269-297 defined by 5′-EEEEEETETNFPEPPQDQESSPIENDSSP-3′ (SEQ ID NO: 110), amino acid residues 268-297 defined by 5′-QEEEEEETETNFPEPPQDQESSPIENDSSP-3′ (SEQ ID NO: 111), amino acid residues 267-297 defined by 5′-IQEEEEEETETNFPEPPQDQESSPIENDSSP-3′ (SEQ ID NO: 112), amino acid residues 266-297 defined by 5′-PIQEEEEEETETNFPEPPQDQESSPIENDSSP-3′ (SEQ ID NO: 113), amino acid residues 265-297 defined by 5′-IPIQEEEEEETETNFPEPPQDQESSPIENDSSP-3′ (SEQ ID NO: 114), amino acid residues 264-297 defined by 5′-IIPIQEEEEEETETNFPEPPQDQESSPIENDSSP-3′ (SEQ ID NO: 115), amino acid residues 263-297 defined by 5′-EIIPIQEEEEEETETNFPEPPQDQESSPIENDSSP-3′ (SEQ ID NO: 116), amino acid residues 262-297 defined by 5′-IEIIPIQEEEEEETETNFPEPPQDQESSPIENDSSP-3′ (SEQ ID NO: 117), amino acid residues 261-297 defined by 5′-DIEIIPIQEEEEEETETNFPEPPQDQESSPIENDSSP-3′ (SEQ ID NO: 118), amino acid residues 260-297 defined by 5′-EDIEIIPIQEEEEEETETNFPEPPQDQESSPIENDSSP-3′ (SEQ ID NO: 119), amino acid residues 259-297 defined by 5′-EEDIEIIPIQEEEEEETETNFPEPPQDQESSPIENDSSP-3′ (SEQ ID NO: 120), amino acid residues 258-297 defined by 5′-NEEDIEIIPIQEEEEEETETNFPEPPQDQESSPIENDSSP-3′ (SEQ ID NO: 121), amino acid residues 257-297 defined by 5′-KNEEDIEIIPIQEEEEEETETNFPEPPQDQE SSPIENDSSP-3′ (SEQ ID NO: 122), amino acid residues 256-297 defined by 5′-PKNEEDIEIIPIQEEEEEETETNFPEPPQDQESSPIENDSSP-3′ (SEQ ID NO: 123), amino acid residues 255-297 defined by 5′-QPKNEEDIEIIPIQEEEEEETETNFPEPPQDQESSPIENDSSP-3′ (SEQ ID NO: 124), amino acid residues 254-297 defined by 5′-SQPKNEEDIEIIPIQEEEEEETETNFPEPPQDQESSPIENDSSP-3′ (SEQ ID NO: 125), amino acid residues 253-297 defined by 5′-SSQPKNEEDIEIIPIQEEEEEETETNFPEPPQDQESSPIENDSSP-3′ (SEQ ID NO: 126), amino acid residues 252-297 defined by 5′-TSSQPKNEEDIEIIPIQEEEEEETETNFPEPPQDQESSPIENDSSP-3′ (SEQ ID NO: 127), amino acid residues 251-297 defined by 5′-ETSSQPKNEEDIEIIPIQEEEEEETETNFPEPPQDQESSPIENDSSP-3′ (SEQ ID NO: 128), amino acid residues 250-297 defined by 5′-TETSSQPKNEEDIEIIPIQEEEEEETETNFPEPPQDQESSPIENDSSP-3′ (SEQ ID NO: 129), amino acid residues 249-297 defined by 5′-LTETSSQPKNEEDIEIIPIQEEEEEETETNFPEPPQDQES SPIENDSSP-3′ (SEQ ID NO: 130), amino acid residues 248-297 defined by 5′-GLTETSSQPKNEEDIEIIPIQEEEEEETETNFPEPPQDQESSPIENDSSP-3′ (SEQ ID NO: 131), amino acid residues 247-297 defined by 5′-VGLTETSSQPKNEEDIEIIPIQEEEEEETETNFPEP PQDQESSPIENDSSP-3′ (SEQ ID NO: 132), amino acid residues 246-297 defined by 5′-VVGLTETSSQPKNEEDIEIIPIQEEEEEETETNFPEPPQDQESSPIENDSSP-3′ (SEQ ID NO: 133), amino acid residues 245-297 defined by 5′-EVVGLTETSSQPKNEEDIEIIPIQEEEEEETETNFPEP PQDQESSPIENDSSP-3′ (SEQ ID NO: 134), amino acid residues 244-297 defined by 5′-EEVVGLTETSSQPKNEEDIEIIPIQEEEEEETETNFPEPPQDQESSPIENDSSP-3′ (SEQ ID NO: 135), amino acid residues 243-297 defined by 5′-KEEVVGLTETSSQPKNEEDIEIIPIQEEEEEETETN FPEPPQDQESSPIENDSSP-3′ (SEQ ID NO: 136), amino acid residues 242-297 defined by 5′-IKEEVVGLTETSSQPKNEEDIEIIPIQEEEEEETETNFPEPPQDQESSPIENDSSP-3′ (SEQ ID NO: 137), amino acid residues 241-297 defined by 5′-EIKEEVVGLTETSSQPKNEEDIEIIPIQEE EEEETETNFPEPPQDQESSPIENDSSP-3′ (SEQ ID NO: 138), amino acid residues 240-297 defined by 5′-IEIKEEVVGLTETSSQPKNEEDIEIIPIQEEEEEETETNFPEPPQDQESSPIENDSSP-3′ (SEQ ID NO: 139), amino acid residues 239-297 defined by 5′-TIEIKEEVVGLTETSSQPKN EEDIEIIPIQEEEEEETETNFPEPPQDQESSPIENDSSP-3′ (SEQ ID NO: 140), amino acid residues 238-297 defined by 5′-QTIEIK EEVVGLTETSSQPKNEEDIEIIPIQEEEEEETETNFPEPPQDQESS PIENDSSP-3′ (SEQ ID NO: 141), amino acid residues 237-297 defined by 5′-EQTIEIKEEVVGLTETSSQPKN EEDIEIIPIQEEEEEETETNFPEPPQDQESSPIENDSSP-3′ (SEQ ID NO: 142), amino acid residues 236-297 defined by 5′-KEQTIEIKEEVVGLTETSSQPK NEEDIEIIPIQEEEEEETETNFPEPPQDQESSPIENDSSP-3′ (SEQ ID NO: 143), amino acid residues 235-297 defined by 5′-KKEQTIEIKEEVVGLTETSSQPKNEEDIEIIPIQEEEEEETETNFP EPPQDQESSPIENDSSP-3′ (SEQ ID NO: 144), amino acid residues 234-297 defined by 5′-EKKEQTIEIKEEVVGLTETSSQPKNEEDIEIIPIQEEEEEETETNFPEPPQDQESSPIENDSSP-3′ (SEQ ID NO: 145), amino acid residues 233-297 defined by 5′-EEKKEQTIEIKEEVVGLTETSSQPKNEEDIEIIPIQEEEEEETETNFPEPPQDQESSPIENDSSP-3′ (SEQ ID NO: 146), amino acid residues 232-297 defined by 5′-AEEKKEQTIEIKEEVVGLTETSSQPKNEEDIEIIPIQEEEEEETETNFPEPPQDQESSPIENDSSP-3′ (SEQ ID NO: 147), amino acid residues 231-297 defined by 5′-SAEEKKEQTIEIKEEVVGLTETSSQPKNEEDIEIIPIQEEEEEETETNFPEPPQDQESSPIENDSSP-3′ (SEQ ID NO: 148), amino acid residues 230-297 defined by 5′-LSAEEKKEQTIEIKEEVVGLTETSSQPKNEEDIEIIPIQEEEEEETETNFPEPPQDQESSPIENDSSP-3′ (SEQ ID NO: 149), amino acid residues 229-297 defined by 5′-LLSAEEKKEQTIEIKEEVVGLTETSSQPKNEEDIEIIPIQEEEEEETETNFPEPPQDQESSPIENDSSP-3′ (SEQ ID NO: 150), amino acid residues 228-297 defined by 5′-VLLSAEEKKEQTIEIKEEVVGLTETSSQPKNEEDIEIIPIQEEEEEETETNFPEPPQDQESSPIENDSSP-3′ (SEQ ID NO: 151), amino acid residues 227-297 defined by 5′-IVLLSAEEKKEQTIEIKEEVVGLTETSSQPKNEEDIEIIPIQEEEEEETETNFPEPPQDQESSPIENDSSP-3′ (SEQ ID NO: 152), amino acid residues 226-297 defined by 5′-NIVLLSAEEKKEQTIEIKEEVVGLTETSSQPKNEEDIEIIPIQEEEEEETETNFPEPPQDQESSPIENDSSP-3′ (SEQ ID NO: 153), amino acid residues 225-297 defined by 5′-SNIVLLSAEEKKEQTIEIKEEVVGLTETSSQPKNEEDIEIIPIQEEEEEETETNFPEPPQDQESSPIENDSSP-3′ (SEQ ID NO: 154), amino acid residues 224-297 defined by 5′-KSNIVLLSAEEKKEQTIEIKEEVVGLTETSSQPKNEEDIEIIPIQEEEEEETETNFPEPPQDQESSPIENDSS P-3′ (SEQ ID NO: 155), amino acid residues 223-297 defined by 5′-PKSNIVLLSAEEKKEQTIEIKEEVVGLTETSSQPKNEEDIEIIPIQEEEEEETETNFPEPPQDQESSPIENDS SP-3′ (SEQ ID NO: 156), amino acid residues 222-297 defined by 5′-RPKSNIVLLSAEEKKEQTIEIKEEVVGLTETSSQPKNEEDIEIIPIQEEEEEETETNFPEPPQDQESSPIEND SSP-3′ (SEQ ID NO: 157), amino acid residues 221-297 defined by 5′-SRPKSNIVLLSAEEKKEQTIEIKEEVVGLTETSSQPKNEEDIEIIPIQEEEEEETETNFPEPPQDQESSPIEN DSSP-3′ (SEQ ID NO: 158), amino acid residues 220-297 defined by 5′-CSRPKSNIVLLSAEEKKEQTIEIKEEVVGLTETSSQPKNEEDIEIIPIQEEEEEETETNFPEPPQDQESSPIE NDSSP-3′ (SEQ ID NO: 159), amino acid residues 219-297 defined by 5′-TCSRPKSNIVLLSAEEKKEQTIEIKEEVVGLTETSSQPKNEEDIEIIPIQEEEEEETETNFPEPPQDQESSPI ENDSSP-3′ (SEQ ID NO: 160), amino acid residues 218-297 defined by 5′-RTCSRPKSNIVLLSAEEKKEQTIEIKEEVVGLTETSSQPKNEEDIEIIPIQEEEEEETETNFPEPPQDQESSP IENDSSP-3′ (SEQ ID NO: 161), amino acid residues 217-297 defined by 5′-KRTCSRPKSNIVLLSAEEKKEQTIEIKEEVVGLTETSSQPKNEEDIEIIPIQEEEEEETETNFPEPPQDQES SPIENDSSP-3′ (SEQ ID NO: 162), amino acid residues 216-297 defined by 5′-WKRTCSRPKSNIVLLSAEEKKEQTIEIKEEVVGLTETSSQPKNEEDIEIIPIQEEEEEETETNFPEPPQDQE SSPIENDSSP-3′ (SEQ ID NO: 163), amino acid residues 215-297 defined by 5′-EWKRTCSRPKSNIVLLSAEEKKEQTIEIKEEVVGLTETSSQPKNEEDIEIIPIQEEEEEETETNFPEPPQDQ ESSPIENDSSP-3′ (SEQ ID NO: 164), amino acid residues 214-297 defined by 5′-NEWKRTCSRPKSNIVLLSAEEKKEQTIEIKEEVVGLTETSSQPKNEEDIEIIPIQEEEEEETETNFPEPPQD QESSPIENDSSP-3′ (SEQ ID NO: 165), amino acid residues 213-297 defined by 5′-ENEWKRTCSRPKSNIVLLSAEEKKEQTIEIKEEVVGLTETSSQPKNEEDIEIIPIQEEEEEETETNFPEPPQ DQESSPIENDSSP-3′ (SEQ ID NO: 166), amino acid residues 212-297 defined by 5′-VENEWKRTCSRPKSNIVLLSAEEKKEQTIEIKEEVVGLTETSSQPKNEEDIEIIPIQEEEEEETETNFPEPP QDQESSPIENDSSP-3′ (SEQ ID NO: 167), amino acid residues 211-297 defined by 5′-IVENEWKRTCSRPKSNIVLLSAEEKKEQTIEIKEEVVGLTETSSQPKNEEDIEIIPIQEEEEEETETNFPEPP QDQESSPIENDSSP-3′ (SEQ ID NO: 168), and amino acid residues 210-297 defined by 5′-GIVENEWKRTCSRPKSNIVLLSAEEKKEQTIEIKEEVVGLTETSSQPKNEEDIEIIPIQEEEEEETETNFPEP PQDQESSPIENDSSP-3′ (SEQ ID NO: 169).

In another example according to this and other aspects of the present invention, the at least one mutation associated with feature (ii) causes truncation of amino acid residues 253-297 set forth in SEQ ID NO: 1, namely 5′-SSQPKNEEDIEIIPIQEEEEEETETNFPEPPQDQESSPIENDSSP-3′ (SEQ ID NO: 126).

In yet another example according to this and other aspects of the present invention, the at least one mutation to a threonine or a serine located within amino acid residues 1-56 set forth in SEQ ID NO: 1 comprises mutation of T2, T3, S7, T11, S25, S35, S36, T41, S43, S49 and T51.

In yet another example the at least one mutation to a threonine or a serine located within amino acid residues 210-297 set forth in SEQ ID NO: 1 comprises mutation of T83, S221, S225, S231, T239, T250, T252, S253, S254, T275, T277 S288, S289, S295 and S296.

To reduce the number of potential mutagenesis targets, thus reducing alterations in protein structure, Applicants employed three different predictors to select potential phosphorylation sites for modification: (i) NetPhos3.1, which predicts S, T or Y phosphorylation sites in eukaryotic proteins using ensembles of neural networks [22]; (ii) the published crystal structure of the CD20 and Rituximab complex [23]; and (iii) sequence identity between human and mouse CD20, based on the hypothesis that important functional residues will be conserved (BLASTp).

This approach identified seven (7) serine and threonine residues in the N-terminal cytoplasmic domain of CD20 (“C₁”). These residues are represented in bold in the sequence set forth in SEQ ID NO: 11 below:

[SEQ ID NO: 11]
MTTPRNSVNGTFPAEPMKGPIAMQSGPKPLFRRMSSLVGPTQSFFMRES
KTLGAVQ

This approach also identified three (3) serine and threonine residues in the C-terminal cytoplasmic domain of CD20 (“C₃”). These residues are also represented in bold in the sequence set forth in SEQ ID NO: 12 below:

[SEQ ID NO: 12]
GIVENEWKRTCSRPKSNIVLLSAEEKKEQTIEIKEEVVGLTETSSQPKN
EEDIEIIPIQEEEEEETETNFPEPPQDQESSPIENDSSP

Accordingly, in another example according to this and other aspects of the present invention, the at least one mutation associated with feature (iii) comprises mutation of at least one amino acid residue selected from T2, T3, S7, T11, S35, S36 and T51 as set forth in SEQ ID NO: 1.

In yet another example according to this aspect of the present invention, the at least one mutation associated with feature (iv) comprises mutation of at least one amino acid residue selected from S225, S231 and T239 as set forth in SEQ ID NO: 1.

In yet another example according to this and other aspects of the present invention, the at least one mutation associated with feature (v) comprises:
feature (i) and feature (iv);
feature (ii) and feature (iii);
feature (i) and a mutation to S225 of SEQ ID NO: 1;
feature (i) and a mutation to S231 of SEQ ID NO: 1;
feature (i) and a mutation to T239 of SEQ ID NO: 1;
feature (i) and a mutation to S225 and S231 of SEQ ID NO: 1;
feature (i) and a mutation to S225 and T239 of SEQ ID NO: 1;
feature (i) and a mutation to S231 and T239 of SEQ ID NO: 1;
feature (i) and a mutation to S225, S231 and T239 of SEQ ID NO: 1;
feature (ii) and a mutation to T2 of SEQ ID NO: 1;
feature (ii) and a mutation to T3 of SEQ ID NO: 1;
feature (ii) and a mutation to S7 of SEQ ID NO: 1;
feature (ii) and a mutation to T11 of SEQ ID NO: 1;
feature (ii) and a mutation to S35 of SEQ ID NO: 1;
feature (ii) and a mutation to S36 of SEQ ID NO: 1;
feature (ii) and a mutation to T51 of SEQ ID NO: 1;
feature (ii) and a mutation to T2 and T3 of SEQ ID NO: 1;
feature (ii) and a mutation to T2 and S7 of SEQ ID NO: 1;
feature (ii) and a mutation to T2 and T11 of SEQ ID NO: 1;
feature (ii) and a mutation to T2 and S35 of SEQ ID NO: 1;
feature (ii) and a mutation to T2 and S36 of SEQ ID NO: 1;
feature (ii) and a mutation to T2 and T51 of SEQ ID NO: 1;
feature (ii) and a mutation to T3 and S7 of SEQ ID NO: 1;
feature (ii) and a mutation to T3 and T11 of SEQ ID NO: 1;
feature (ii) and a mutation to T3 and S35 of SEQ ID NO: 1;
feature (ii) and a mutation to T3 and S36 of SEQ ID NO: 1;
feature (ii) and a mutation to T3 and T51 of SEQ ID NO: 1;
feature (ii) and a mutation to S7 and T11 of SEQ ID NO: 1;
feature (ii) and a mutation to S7 and S35 of SEQ ID NO: 1;
feature (ii) and a mutation to S7 and S36 of SEQ ID NO: 1;
feature (ii) and a mutation to S7 and T51 of SEQ ID NO: 1;
feature (ii) and a mutation to T11 and S35 of SEQ ID NO: 1;
feature (ii) and a mutation to T11 and S36 of SEQ ID NO: 1;
feature (ii) and a mutation to T11 and T51 of SEQ ID NO: 1;
feature (ii) and a mutation to S35 and S36 of SEQ ID NO: 1;
feature (ii) and a mutation to S35 and T51 of SEQ ID NO: 1;
feature (ii) and a mutation to S36 and T51 of SEQ ID NO: 1;
feature (ii) and a mutation to T2, T3 and S7 of SEQ ID NO: 1;
feature (ii) and a mutation to T2, T3 and T11 of SEQ ID NO: 1;
feature (ii) and a mutation to T2, T3 and S35 of SEQ ID NO: 1;
feature (ii) and a mutation to T2, T3 and S36 of SEQ ID NO: 1;
feature (ii) and a mutation to T2, T3 and T51 of SEQ ID NO: 1;
feature (ii) and a mutation to T2, S7 and T11 of SEQ ID NO: 1;
feature (ii) and a mutation to T2, S7 and S35 of SEQ ID NO: 1;
feature (ii) and a mutation to T2, S7 and S36 of SEQ ID NO: 1;
feature (ii) and a mutation to T2, S7 and T51 of SEQ ID NO: 1;
feature (ii) and a mutation to T2, T11 and S35 of SEQ ID NO: 1;
feature (ii) and a mutation to T2, T11 and S36 of SEQ ID NO: 1;
feature (ii) and a mutation to T2, T11 and T51 of SEQ ID NO: 1;
feature (ii) and a mutation to T2, S35 and S36 of SEQ ID NO: 1;
feature (ii) and a mutation to T2, S35 and T51 of SEQ ID NO: 1;
feature (ii) and a mutation to T2, S36 and T51 of SEQ ID NO: 1;
feature (ii) and a mutation to T3, S7 and T11 of SEQ ID NO: 1;
feature (ii) and a mutation to T3, S7 and S35 of SEQ ID NO: 1;
feature (ii) and a mutation to T3, S7 and S36 of SEQ ID NO: 1;
feature (ii) and a mutation to T3, S7 and T51 of SEQ ID NO: 1;
feature (ii) and a mutation to T3, T11 and S35 of SEQ ID NO: 1;
feature (ii) and a mutation to T3, T11 and S36 of SEQ ID NO: 1;
feature (ii) and a mutation to T3, T11 and T51 of SEQ ID NO: 1;
feature (ii) and a mutation to T3, S35 and S36 of SEQ ID NO: 1;
feature (ii) and a mutation to T3, S35 and T51 of SEQ ID NO: 1;
feature (ii) and a mutation to T3, S36 and T51 of SEQ ID NO: 1;
feature (ii) and a mutation to S7, T11 and S35 of SEQ ID NO: 1;
feature (ii) and a mutation to S7, T11 and S36 of SEQ ID NO: 1;
feature (ii) and a mutation to S7, T11 and T51 of SEQ ID NO: 1;
feature (ii) and a mutation to S7, S35 and S36 of SEQ ID NO: 1;
feature (ii) and a mutation to S7, S35 and T51 of SEQ ID NO: 1;
feature (ii) and a mutation to S7, S36 and T51 of SEQ ID NO: 1;
feature (ii) and a mutation to T11, S35 and S36 of SEQ ID NO: 1;
feature (ii) and a mutation to T11, S35 and T51 of SEQ ID NO: 1;
feature (ii) and a mutation to T11, S36 and T51 of SEQ ID NO: 1;
feature (ii) and a mutation to S35, S36 and T51 of SEQ ID NO: 1;
feature (ii) and a mutation to T2, T3, S7 and T11 of SEQ ID NO: 1;
feature (ii) and a mutation to T2, T3, S7 and S35 of SEQ ID NO: 1;
feature (ii) and a mutation to T2, T3, S7 and S36 of SEQ ID NO: 1;
feature (ii) and a mutation to T2, T3, S7 and T51 of SEQ ID NO: 1;
feature (ii) and a mutation to T2, T3, T11 and S35 of SEQ ID NO: 1;
feature (ii) and a mutation to T2, T3, T11 and S36 of SEQ ID NO: 1;
feature (ii) and a mutation to T2, T3, T11 and T51 of SEQ ID NO: 1;
feature (ii) and a mutation to T2, T3, S35 and S36 of SEQ ID NO: 1;
feature (ii) and a mutation to T2, T3, S35 and T51 of SEQ ID NO: 1;
feature (ii) and a mutation to T2, T3, S36 and T51 of SEQ ID NO: 1;
feature (ii) and a mutation to T2, S7, T11 and S35 of SEQ ID NO: 1;
feature (ii) and a mutation to T2, S7, T11 and S36 of SEQ ID NO: 1;
feature (ii) and a mutation to T2, S7, T11 and T51 of SEQ ID NO: 1;
feature (ii) and a mutation to T2, S7, S35 and S36 of SEQ ID NO: 1;
feature (ii) and a mutation to T2, S7, S35 and T51 of SEQ ID NO: 1;
feature (ii) and a mutation to T2, S7, S36 and T51 of SEQ ID NO: 1;
feature (ii) and a mutation to T2, T11, S35 and S36 of SEQ ID NO: 1;
feature (ii) and a mutation to T2, T11, S35 and T51 of SEQ ID NO: 1;
feature (ii) and a mutation to T2, T11, S36 and T51 of SEQ ID NO: 1;
feature (ii) and a mutation to T2, S35, S36 and T51 of SEQ ID NO: 1;
feature (ii) and a mutation to T3, S7, T11 and S35 of SEQ ID NO: 1;
feature (ii) and a mutation to T3, S7, T11 and S36 of SEQ ID NO: 1;
feature (ii) and a mutation to T3, S7, T11 and T51 of SEQ ID NO: 1;
feature (ii) and a mutation to T3, S7, S35 and S36 of SEQ ID NO: 1;
feature (ii) and a mutation to T3, S7, S35 and T51 of SEQ ID NO: 1;
feature (ii) and a mutation to T3, S7, S36 and T51 of SEQ ID NO: 1;
feature (ii) and a mutation to T3, T11, S35 and S36 of SEQ ID NO: 1;
feature (ii) and a mutation to T3, T11, S35 and T51 of SEQ ID NO: 1;
feature (ii) and a mutation to T3, T11, S36 and T51 of SEQ ID NO: 1;
feature (ii) and a mutation to T3, S35, S36 and T51 of SEQ ID NO: 1;
feature (ii) and a mutation to S7, T11, S35 and S36 of SEQ ID NO: 1;
feature (ii) and a mutation to S7, T11, S35 and T51 of SEQ ID NO: 1;
feature (ii) and a mutation to S7, T11, S36 and T51 of SEQ ID NO: 1;
feature (ii) and a mutation to S7, S35, S36 and T51 of SEQ ID NO: 1;
feature (ii) and a mutation to T11, S35, S36 and T51 of SEQ ID NO: 1;
feature (ii) and a mutation to T2, T3, S7, T11 and S35 of SEQ ID NO: 1;
feature (ii) and a mutation to T2, T3, S7, T11 and S36 of SEQ ID NO: 1;
feature (ii) and a mutation to T2, T3, S7, T11 and T51 of SEQ ID NO: 1;
feature (ii) and a mutation to T2, T3, S7, S35 and S36 of SEQ ID NO: 1;
feature (ii) and a mutation to T2, T3, S7, S35 and T51 of SEQ ID NO: 1;
feature (ii) and a mutation to T2, T3, S7, S36 and T51 of SEQ ID NO: 1;
feature (ii) and a mutation to T2, T3, T11, S35 and S36 of SEQ ID NO: 1;
feature (ii) and a mutation to T2, T3, T11, S35 and T51 of SEQ ID NO: 1;
feature (ii) and a mutation to T2, T3, T11, S36 and T51 of SEQ ID NO: 1;
feature (ii) and a mutation to T2, T3, S35, S36 and T51 of SEQ ID NO: 1;
feature (ii) and a mutation to T2, S7, T11, S35 and S36 of SEQ ID NO: 1;
feature (ii) and a mutation to T2, S7, T11, S35 and T51 of SEQ ID NO: 1;
feature (ii) and a mutation to T2, S7, T11, S36 and T51 of SEQ ID NO: 1;
feature (ii) and a mutation to T2, S7, S35, S36 and T51 of SEQ ID NO: 1;
feature (ii) and a mutation to T2, T11, S35, S36 and T51 of SEQ ID NO: 1;
feature (ii) and a mutation to T3, S7, T11, S35 and S36 of SEQ ID NO: 1;
feature (ii) and a mutation to T3, S7, T11, S35 and T51 of SEQ ID NO: 1;
feature (ii) and a mutation to T3, S7, T11, S36 and T51 of SEQ ID NO: 1;
feature (ii) and a mutation to T3, S7, S35, S36 and T51 of SEQ ID NO: 1;
feature (ii) and a mutation to T3, T11, S35, S36 and T51 of SEQ ID NO: 1;
feature (ii) and a mutation to S7, T11, S35, S36 and T51 of SEQ ID NO: 1;
feature (ii) and a mutation to T2, T3, S7, T11, S35 and S36 of SEQ ID NO: 1;
feature (ii) and a mutation to T2, T3, S7, T11, S35 and T51 of SEQ ID NO: 1;
feature (ii) and a mutation to T2, T3, S7, T11, S35 and S36 of SEQ ID NO: 1;
feature (ii) and a mutation to T2, T3, S7, T11, S36 and T51 of SEQ ID NO: 1;
feature (ii) and a mutation to T2, T3, S7, S35, S36 and T51 of SEQ ID NO: 1;
feature (ii) and a mutation to T2, T3, T11, S35, S36 and T51 of SEQ ID NO: 1;
feature (ii) and a mutation to T2, S7, T11, S35, S36 and T51 of SEQ ID NO: 1;
feature (ii) and a mutation to T3, S7, T11, S35, S36 and T51 of SEQ ID NO: 1;
feature (ii) and a mutation to T2, T3, S7, T11, S35, S36 and T51 of SEQ ID NO: 1; and
features (iii) and (iv) and includes any of the sub-combinations presented immediately above.

In yet another example according to this and other aspects of the present invention, the at least one mutation to a serine or threonine amino acid residue located within amino acid resides 1-56 and 210-297 set forth in SEQ ID NO: 1, respectively, comprises a substitution mutation, a deletion mutation or an insertion mutation.

In a related example according to this and other aspects of the present invention, the at least one mutation to a serine or threonine located within amino acid resides 1-56 and 210-297 set forth in SEQ ID NO: 1, respectively, comprises a conservative or non-conservative substitution comprising a naturally or non-naturally occurring amino acid residue.

Examples of naturally occurring amino acids include, without limitation, alanine (A), arginine (R), asparagine (N), aspartic acid (D), cysteine (C), glutamine (Q), glutamate (E), glycine (G), histidine (H), isoleucine (I), leucine (L), lysine (K), methionine (M), phenylalanine (F), proline (P), serine (S), threonine (T), tryptophan (W), tyrosine (Y) and valine (V). Examples of non-naturally occurring amino acids are reviewed in (47).

Specific examples of the modified CD20 proteins in which intracellular signalling is reduced or abrogated when expressed by a cell are outlined in Table 2 in Example 3. Sequence specific examples include, without limitation, SEQ ID Nos: 2-7 and include a combination of truncation and/or serine/threonine substitution mutations as described herein.

Accordingly, in yet another aspect the present invention provides a modified CD20 protein comprising or consisting in the sequence set forth in any one of SEQ ID NO: 2, SEQ ID NO: 3, SEQ ID NO: 4, SEQ ID NO: 5, SEQ ID NO: 6 or SEQ ID NO: 7, or a variant sequence comprising at least 80%, 85%, 86%, 87%, 88%, 89%, 90%, 91%, 92%, 93%, 94%, 95%, 96%, 97%, 98% and 99% sequence identity to SEQ ID NO: 2, SEQ ID NO: 3, SEQ ID NO: 4, SEQ ID NO: 5, SEQ ID NO: 6 or SEQ ID NO: 7, respectively.

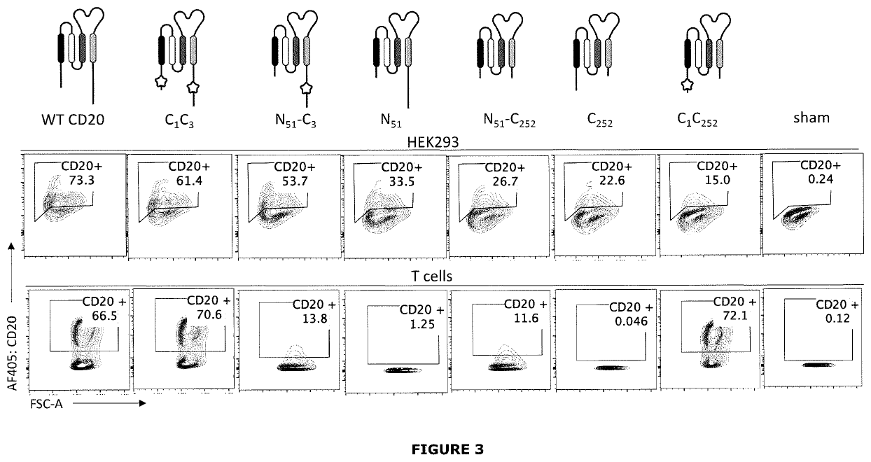

The data presented in Example 3 and FIG. 3 demonstrate that the C₁C₃ (SEQ ID NO: 7) and C₁C₂₅₂ (SEQ ID NO: 5) mutant proteins were attached to, or associated with the membrane of both a HEK293 and a T cell line at levels similar to that observed for wild-type CD20 protein (SEQ ID NO: 1). Accordingly, the C₁C₃ (SEQ ID NO: 7) and C₁C₂₅₂ (SEQ ID NO: 5) mutants were taken forward for further analysis in cell signalling assays.

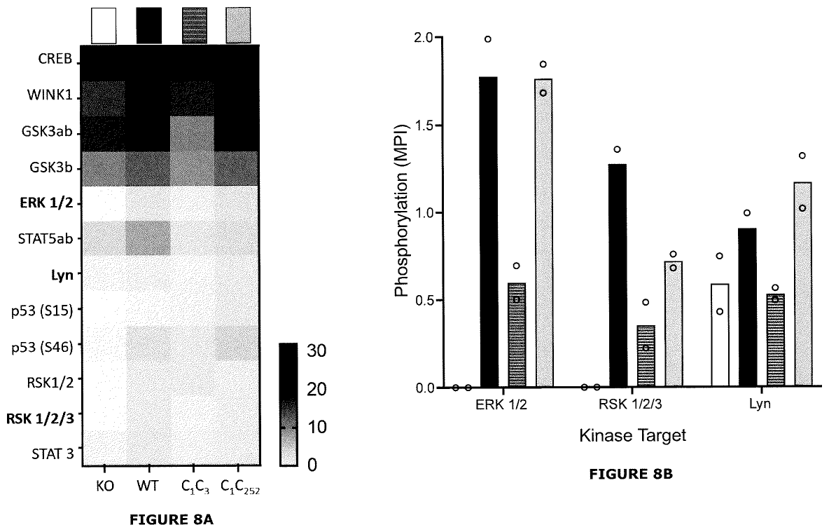

These data are presented in Examples 7 and 8 and further reflects that phosphorylation is likely to play an important role in membrane trafficking and intracellular signalling pathways in CD20⁺ cells. Specifically, in an HG3 CD20KO cell line Rituximab stimulation of C₁C₃ (SEQ ID NO: 7) or C₁C₂₅₂ (SEQ ID NO: 5) induced impaired signalling as evidenced by a difference in phosphorylation of all proteins measured and impaired Ca²⁺ influx (FIGS. 7 and 8).

Accordingly, in a further aspect the present invention provides a modified human CD20 protein comprising:
(i) at least one mutation to a threonine or a serine located within amino acid residues 1-56 set forth in SEQ ID NO: 1; and
(ii) truncation of amino acid residues 253-297 set forth in SEQ ID NO: 1,

wherein the at least one mutation defined by (i) and (ii) causes reduced or abrogated intracellular signalling when the modified CD20 protein is attached to or associated with the membrane of a cell.

In yet a further aspect the present invention provides a modified human CD20 protein comprising:
(i) at least one mutation to a threonine or a serine located within amino acid residues 1-56 set forth in SEQ ID NO: 1; and
(ii) at least one mutation to a threonine or a serine located within amino acid residues 210-297 set forth in SEQ ID NO: 1,

wherein the at least one mutation defined by (i) and (ii) causes reduced or abrogated intracellular signalling when the modified CD20 protein is attached to or associated with the membrane of a cell.

### Mutant (Human) CD20—Cytoplasmic and Extracellular Loop Domain Modifications

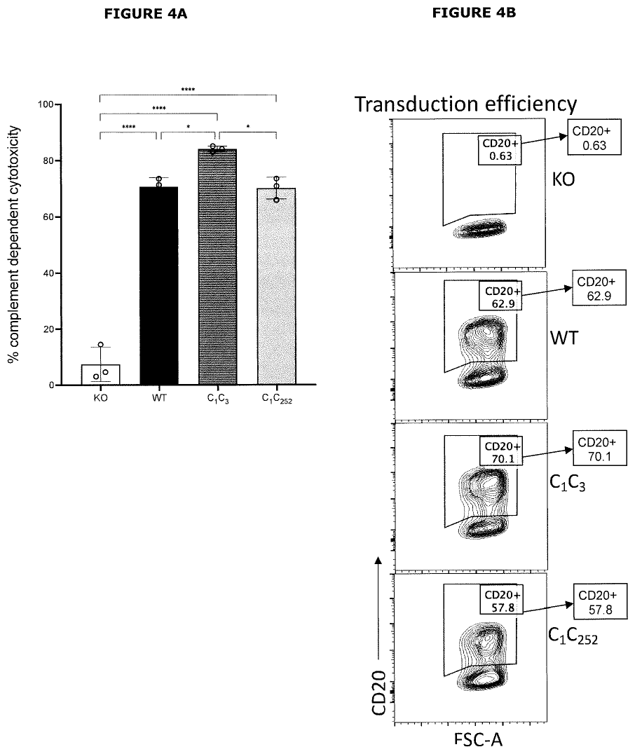

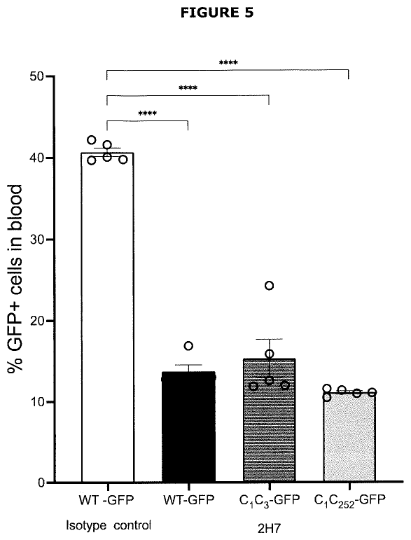

The preliminary data presented in Examples 4 and 5 read on conjunction with FIGS. 4 and 5 demonstrate mutations to predicted phosphorylation sites permit Rituximab induced killing in HG3 cell line and primary T cells which express the modified human CD20 proteins as described herein. Accordingly, transgenes incorporating a nucleic acid encoding the modified human CD20 proteins according to the present invention (e.g.) incorporated for expression in a CAR T-cell construct may be utilised as a genetic biomarker and/or as a safety/suicide switch to avoid cytotoxicity issues in patients who receive CAR T-cell therapies.

Accordingly, the modified human CD20 proteins according to the present invention may be further modified in the extracellular loop to induce changes in the epitope recognition sequence targeted by clinically approved monoclonal antibodies, including without limitation, Rituximab, Obinutuzumab and Ocrelizumab, so as to engineer selective binding (or binding elimination) by these therapeutic molecules. Refer to Example 10.

Accordingly, in yet another aspect of the present invention there is provided a modified human CD20 protein comprising:
(i) at least one mutation causing a truncation to any one or more of amino acid residues 1-56 set forth in SEQ ID NO: 1;
(ii) at least one mutation causing a truncation to any one or more of amino acid residues 210-297 set forth in SEQ ID NO: 1;
(iii) at least one mutation to a threonine or a serine located within amino acid residues 1-56 set forth in SEQ ID NO: 1;
(iv) at least one mutation to a threonine or a serine located within amino acid residues 210-297 set forth in SEQ ID NO: 1;
(v) a combination comprising any one of (i) to (iv); and
(vi) at least one mutation to an amino acid within residues 142-188 as set forth in SEQ ID NO: 1,

wherein any one of (i) to (v) causes reduced or abrogated intracellular signalling when the modified CD20 protein is attached to or associated with the membrane of a cell.

In an example according to this aspect of the present invention, the at least one mutation to an amino acid within amino acid residues 142-188 as set forth in SEQ ID NO: 1-include, without limitation, N173, S174 and N176.

The data presented in Examples 3-5 and referred to immediately above when read in conjunction with the data presented in FIG. 7 facilitate the identification of specific constructs which meet this purpose (i.e.) constructs which incorporate mutations to the cytoplasmic domain defined by C₁-C₃ and C₁-C₂₅₂ in addition to mutations to the extracellular loop domain.

Accordingly, in yet a further aspect the present invention provides a modified human CD20 protein comprising:
(i) at least one mutation to T2, T3, S7, T11, S35, S36 and T51 as set forth in SEQ ID NO: 1;
(ii) at least one mutation to N173, S174 and N176 as set forth in SEQ ID NO: 1; and
(iii) truncation of amino acid residues 253-297 as set forth in SEQ ID NO: 1.

In yet another aspect the present invention provides a modified human CD20 protein comprising:
(i) at least one mutation to T2, T3, S7, T11, S35, S36 and T51 as set forth in SEQ ID NO: 1;
(ii) at least one mutation to N173, S174 and N176 as set forth in SEQ ID NO: 1; and
(iii) at least one mutation to S225, S231 and T239 as set forth in SEQ ID NO: 1.

### Expression of Mutant (Human) CD20

The modified human CD20 proteins according to the present invention may be expressed by one or more cells, in isolation or within a cell population, for utility as (e.g.) a transgene selection marker comprising the modified human CD20 protein or as a suicide/safety switch to manage adverse effects in a of a therapy involving genetic transduction or transfection of cells or as a marker to facilitate the detection of the modified cells within recipients of the therapy.

As such, in yet another aspect the present invention provides a cell expressing a modified human CD20 protein as described herein, wherein the modified human CD20 protein traffics to and is attached to or associated with the membrane of the cell.

In an example according to this and other aspects of the present invention, the cell is selected from T-cell, a natural killer (NK) cell, a B-cell, a myeloid cell, a pluripotent stem cell, a non-haematopoietic cell line and a haematopoietic stem cell.

The present invention further contemplates specific cell lines, such as a haematopoietic stem cell line or a leukaemia-derived cell line which has been modified to express the human CD20 proteins described herein. For example, one method of generating ‘off the shelf’ CAR therapies involves taking a non-haematopoietic cell line (e.g. K562), and genetically modifying it to reduce alloreactivity and express a CAR as a therapeutic agent.

### Nucleic Acids Encoding Mutant Human CD20

Also contemplated by the present invention are nucleic acids (e.g.) deoxyribose nucleic acid (DNA), messenger ribose nucleic acid (mRNA) or a complementary deoxyribose nucleic acid (cDNA) which encode a modified human CD20 protein as described herein.

Accordingly, in yet another aspect the present invention provides an isolated nucleic acid molecule encoding any modified human CD20 protein as described herein.

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (e.g. [24]).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (e.g. [48, 49]).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers [50]. However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one example, the nucleic acids introduced into a cell (e.g. a T-cell) are RNA. In another example, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a modified or chimeric membrane protein such as the modified human CD20 proteins described herein.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one example, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one example, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one example, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other examples the 5′ UTR can be derived from an RNA virus whose RNA genome is stable in cells. In other examples various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one example, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one example, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatemeric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (e.g. [25, 26]).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one example, the poly(A) tail is between 100 and 5000 adenosines.

Poly(A) tails of RNAs can be further extended following in vitro transcription with the use of a poly(A) polymerase, such as E. coli polyA polymerase (E-PAP). In one example, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred example, RNAs produced by the methods disclosed herein include a 5′ cap. The 5′ cap is provided using techniques known in the art and described herein (e.g. [59]).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some examples, the RNA encoding a transgene is electroporated into the cells. In one example, the RNA encoding the transgene is in vitro transcribed RNA.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end.

Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid-based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in [53]. The various parameters including electric field strength required for electroporation of any known cell type are generally known in the relevant research literature as well as numerous patents and applications in the field [54]. Apparatus for therapeutic application of electroporation are available commercially, e.g., the MedPulser™ DNA Electroporation Therapy System (Inovio/Genetronics, San Diego, Calif.), and are described in patents such as [55]; electroporation may also be used for transfection of cells in vitro as described e.g. [56]. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

The invention is further described with reference to the following examples. It will be appreciated that the invention as claimed is not intended to be limited in any way by these examples.

### EXAMPLES

### Example 1: Methodology

### Plasmids Construction and Bacterial Strain

All gene fragments were designed in silico using Benchling (Benchling 2022), synthesized de novo by IDT (Integrated DNA Technologies, Inc). Fragments for testing in human cell lines or primary cells were cloned into a LeGO plasmid backbone [27] using PmeI/BamHI restriction enzymes (New England Biolabs) under the EF1α promoter. Fragments for testing in mouse cell lines or primary cells were cloned into a pmX_GFP plasmid backbone using XhoI/PacI enzymes. Plasmids were propagated in chemically competent Stable3 E. coli (F-mcrB mrrhsdS20(r_{B}⁻, m_{B}⁻) recA13 supE44 ara-14 ga/K2 lacY1 proA2 rpsL20(Str^{R}) xyl-5 λ⁻ leumtl-1) (ThermoFisher Scientific).

### Cell Lines and Primary Cells

### HEK293

HEK293 cell line for screening of CD20 truncations/mutations and lentivirus packaging was obtained from Thermofisher Scientific.

### HG3

HG3 B-cell line CD20 knock-out (CD20KO) was generated and kindly provided by Michal Smida's laboratory in the Central European Institute of Technology, along with the CD20+ HG3 control cell line.

### Platinum-E

Platinum-E cells, a retroviral packaging cell line was obtained from Cell Biolabs.

Human Primary T cells

Primary T cells were isolated from buffy coats obtained from the New Zealand Blood Service (University of Otago Human Ethics Committee (Health) H20-173). Peripheral blood mononuclear cells (PBMCs) were isolated using SepMate columns and Lymphoprep (Stemcell Technologies) and T cells were selected and activated for 24 hours using anti-CD3/CD28 Dynabeads (Thermo Fisher Scientific, 11141D) before transduction.

### HEK295FT Transfection

For all experiments screening for CD20 detection on the cell membrane, HEK293 cells were transfected with LeGO_CD20 constructs using lipofectamine 3000 (ThermoFisher Scientific, L3000015).

### Lentiviral Production

Lentiviral particles were packaged in HEK293 cells transfected with LeGO_CD20, and 3^{rd} generation lentivirus packaging/structural plasmids pMD2-G, pMDLg and pRSV (Adgene). Forty-eight hours after transfection lentiviral particles were harvested from the growth media by centrifugation and frozen at −70° C. until use.

### Human B Cell Transduction

HG3 cell line was transduced with lentiviral particles containing CD20 phospho-mutations/truncations by spinfection using polybrene (Sigma-Aldrich, TR-1003-G).

### Flow Cytometry

All flow cytometry experiments were performed in a five laser (UV-V-B-YG-R) Cytek Aurora Flow Cytometer.

### Detection of Surface CD20

Surface CD20 was detected using 2H7 conjugated to APC/Fire 750 (Biolegend, 302357), Rituximab conjugated to Alexa Fluor 405 (Novus Biologicals), Obintuzumab conjugated to APC (Leinco, LT907) or BD Quantibrite™ PE Mouse Anti-Human CD20 (L27-PE) (BD Biosciences, 347201).

### In Vitro CD20 Mediated Killing

CD20KO HG3 cells and CD20KO HG3 cells transduced with CD20WT, C₁C₃ or C₁C₂₅₂ constructs were incubated in a 96 U bottom well plate with 10 ug Rituximab (Abcam, ab275973) per mL of serum free RPMI for 30 min at 37° C. Cells were washed to remove unbound Rituximab and incubated for 1.5 hours with 25% baby rabbit complement (BioRad, C12CA) diluted in RPMI. Then, cells were stained with Zombie NIR (Biolegend, BIO0423105) and cell viability assessed by flow cytometry.

### Mouse T-Cell Transduction

Retrovirus containing pmX_hCD20WT-GFP, pmX_C₁C₃-GFP or pmX_C₁C₂₅₂-GFP was packaged in Platinum-E cells and used to transduce T cells isolated from C57 mice (B6.SJL-Ptprc^{a}, CD45.2+). Five million transduced cells were transferred intravenously to each mouse (B6.SJL-Ptprc^{b}, CD45.1+), and a control group was given sham transduced cells. Each group contained 5 mice.

### In Vivo CD20+ T Cell Depletion

One day after adoptive transfer, hCD20WT-GFP, C₁C₃-GFP, C₁C₂₅₂-GFP and sham groups were given 500 μg of 2H7 mouse anti-human CD20 antibody (BioXCell, BE0276) intraperitoneally and a second injection of 250 μg 2H7 the following day. As a control, one group of mice who received hCD20WT-GFP T-cells received the same doses of an isotype control antibody (BioXCell, BE0086). One day after second dose, 200 μL of blood was collected from each mouse (Animal Ethics Committee 30148). Lymphocytes where purified and stained with anti CD45.1-APC Fire750 (Biolegend, 110752), anti CD19-EF450 (eBioscience, 48-0193-82), anti TCRbeta-PE (BD, 553172) and Zombie NIR (Biolegend, BIO0423105). GFP production was used as a surrogate for hCD20 expression. Cells were analysed by flow cytometry to assess percentage of adoptively transferred GFP+ T cells.

### CD20+ Cell Sorting

CD20KO HG3 cells transduced with WT, C₁C₃ and C₁C₂₅₂ CD20 were selected using the EasySep™ PE Positive Selection Kit following the manufacturer's instructions. Briefly, non-specific antibody binding sites where blocked and cells where incubated with BD™ PE Mouse Anti-Human CD20. Selection cocktail was added before incubating samples with RapidSpheres™ and proceeding to magnetic selection with an EasyEights™ magnet. Selected cells were expanded in RPMI medium supplemented with 10% FBS and 1% Penicillin/Streptomycin (complete RPMI) at 37° C. and 5% CO₂ and frozen in liquid nitrogen until use.

### Evaluation of CD20 Mediated Signalling

### Ca²⁺ Flux Assay

Intracellular Ca²⁺ mobilization induced by Rituximab crosslinking was measured by flow cytometry using the Calcium Flux Assay Kit (Abcam, 233472). Selected WT, C₁C₃, C₁C₂₅₂ transduced and untransduced CD20KO HG3 cells where stained with Zombie NIR and 520 AM Ca²⁺ dyes at 37° C. for 10 minutes. Cells where washed and non-specific antibody binding sites blocked before incubating with 30 μg/mL Rituximab (RIXIMYO®) for 30 min. Unbound Rituximab was washed off and cells and buffers where rested for 45 min at 37° C. Basal Ca²⁺ related fluorescence intensity was measured, and 200 μg/mL of goat anti-human IgG hyper-crosslinking antibody (Invitrogen, 62-8400) added to measure Peak Ca²⁺ related fluorescence intensity.

### CD20 Mediated Phosphorylation

One million HG3 CD20KO cells transduced with CD20 mutants were stimulated with g/mL Rituximab (RIXIMYO, 2583917) for 24 hours. CD20 mediated phosphorylation was evaluated using the Proteome Profiler Human Phospho-Kinase Array Kit (R&D systems, ARY003C) and pixel intensity of each array dot was analysed using Image Lab software as per the manufacturer's instructions.

### Statistical Analysis

Data were analysed using GraphPad Prism 10. Values are expressed as mean±standard error of mean (SEM). Statistical significance was assessed using a multiple comparison 2way ANOVA and considered at p<0.05.

### Example 2: Truncated/Chimeric CD20 not Detected on Cell Membrane

The initial strategy to the design of a modified CD20 molecule with abrogated intracellular signalling was to (i) truncate large portions of the cytoplasmic domains and (ii) combine the cytoplasmic and transmembrane domains of CD20. The various sequence constructs developed for these initial experiments are presented in Table 1 and include the constructs designated CD20t, CD20raft, CD20loop, CD20raft v2, CD20loop v2, CD20t v3.1 and CD20t v3.2.

Since significant modifications to the membrane spanning 4a (MS4a) protein may affect the protein structure and its ability to traffic to and be correctly attached to or associated with the cell membrane, applicants sought to interrogate the effect of truncating different CD20 domains.

TABLE 1
Summary of CD20 truncated/chimeric constructs not detected on cell
membrane

| CD20 | | |
|---|---|---|
| construct | Description | Sequence |
| CD20WT | wild type amino acidic sequence | MTTPRNSVNGTFPAEPMKGPIAMQSGPKPLFR |
| | of human CD20 | RMSSLVGPTQSFFMRESKTLGAVQIMNGLFHI |
| | | ALGGLLMIPAGIYAPICVTVWYPLWGGIMYIIS |
| | | GSLLAATEKNSRKCLVKGKMIMNSLSLFAAIS |
| | | GMILSIMDILNIKISHFLKMESLNFIRAHTPYIN |
| | | IYNCEPANPSEKNSPSTQYCYSIQSLFLGILSV |
| | | MLIFAFFQELVIAGIVENEWKRTCSRPKSNIVL |
| | | LSAEEKKEQTIEIKEEVVGLTETSSQPKNEEDI |
| | | EIIPIQEEEEEETETNFPEPPQDQESSPIENDSS |
| | | P [SEQ ID NO: 1] |
| CD20t | truncated 1^{st} cytoplasmic tail | MRESKTLGAVQIMNGLFHIALGGLLMIPAGIYA |
| | M46-A209 + truncated 3rd | PICVTVWYPLWGGIMYIISGSLLAATEKNSRK |
| | cytoplasmic tail S253-P297 | CLVKGKMIMNSLSLFAAISGMILSIMDILNIKI |
| | | SHFLKMESLNFIRAHTPYINIYNCEPANPSEKN |
| | | SPSTQYCYSIQSSSQPKNEEDIEIIPIQEEEEEE |
| | | TETNFPEPPQDQESSPIENDSSP [SEQ ID |
| | | NO: 14] |
| CD20raft | completely truncated 1^{st} | MNGLFHIALGGLLMIPAGIYAPICVTVWYPLW |
| | cytoplasmic tail M58-S225, to | GGIMYIISGSLLAATEKNSRKCLVKGKMIMNS |
| | include lipid raft localisation | LSLFAAISGMILSIMDILNIKISHFLKMESLNFI |
| | signal | RAHTPYINIYNCEPANPSEKNSPSTQYCYSIQS |
| | | LFLGILSVMLIFAFFQELVIAGIVENEWKRTCS |
| | | RPKS [SEQ ID NO: 15] |
| CD20loop | 2^{nd} extracellular loop only: K114- | MKGKMIMNSLSLFAAISGMILSIMDILNIKISH |
| | A209 + truncated 3rd | FLKMESLNFIRAHTPYINIYNCEPANPSEKNSP |
| | cytoplasmic tail S253-P297 | STQYCYSIQSLFLGILSVMLIFAFFQELVIASQP |
| | | KNEEDIEIIPIQEEEEEETETNFPEPPQDQESSP |
| | | IENDSP [SEQ ID NO: 16] |
| CD20raft | same as CD20raft but starting at | MRESKTLGAVQMNGLFHIALGGLLMIPAGIYA |
| v2 | M46 | PICVTVWYPLWGGIMYIISGSLLAATEKNSRK |
| | | CLVKGKMIMNSLSLFAAISGMILSIMDILNIKI |
| | | SHFLKMESLNFIRAHTPYINIYNCEPANPSEKN |
| | | SPSTQYCYSIQSLFLGILSVMLIFAFFQELVIAG |
| | | IVENEWKRTCSRPKS [SEQ ID NO: 17] |
| CD20loop | same as v2, starting at K106 | MKNSRKCLVKGKMIMNSLSLFAAISGMILSIM |
| v2 | | DILNIKISHFLKMESLNFIRAHTPYINIYNCEPA |
| | | NPSEKNSPSTQYCYSIQSLFLGILSVMLIFAFF |
| | | QELVIASQPKNEEDIEIIPIQEEEEEETETNFPE |
| | | PPQDQESSPIENDSSP [SEQ ID NO: 18] |
| CD20t | cytoplasmic domain 1- | MTTPRNSVNGTFPAEPMKGPIAMQSGPKPLFR |
| v3.1 | transmembrane 1-extracellular | RMSSLVGPTQSFFMRESKTLGAVQIMNGLFHI |
| | 2-transmembrane 4- | ALGGLLMIPAGIYAKISHFLKMESLNFIRAHTP |
| | cytoplasmic 3 | YINIYNCEPANPSEKNSPSTQYCYSIQSLFLGIL |
| | | SVMLIFAFFQELVIAGIVENEWKRTCSRPKSNI |
| | | VLLSAEEKKEQTIEIKEEVVGLTETSSQPKNEE |
| | | DIEIIPIQEEEEEETETNFPEPPQDQESSPIEND |
| | | SSP [SEQ ID NO: 19] |
| CD20t | cytoplasmic 1-transmembrane 3- | MTTPRNSVNGTFPAEPMKGPIAMQSGPKPLFR |
| v3.2 | extracellular 2-transmembrane | RMSSLVGPTQSFFMRESKTLGAVQSLSLFAAI |
| | 4-cytoplasmic 3 | SGMILSIMDILNIKISHFLKMESLNFIRAHTPYI |
| | | NIYNCEPANPSEKNSPSTQYCYSIQSLFLGILS |
| | | VMLIFAFFQELVIAGIVENEWKRTCSRPKSNIV |
| | | LLSAEEKKEQTIEIKEEVVGLTETSSQPKNEED |
| | | IEIIPIQEEEEEETETNFPEPPQDQESSPIENDS |
| | | SP [SEQ ID NO: 20] |
| CD20t | GM-CSFR signal peptide-CD20 | MLLLVTSLLLCELPHPAFLLIPNIYNCEPANPSE |
| v4 | extracellular 163-187- | KNSPSTQYCYSIQIATGMVGALLLLLVVALGIG |
| | transmembrane EGFR | LFM [SEQ ID NO: 21] |
| CD20t | GM-CSFR signal peptide-CD20 | MLLLVTSLLLCELPHPAFLLIPCEPANPSEKNSP |
| v5.1 | extracellular 167-187- | STQYCYSIQIATGMVGALLLLLVVALGIGLFM |
| | transmembrane EGFR | [SEQ ID NO: 22] |
| CD20t | CD28 signal peptid-CD20 | MLRLLLALNLFPSIQVTGCEPANPSEKNSPSTQ |
| v5.2 | extracellular 167-188-CD28 | YCYSIQSFWVLVVVGGVLACYSLLVTVAFIIFW |
| | transmembrane-CD28 | VRSKRSRLLHSDYMNMTPRRPGPTRKHYQPY |
| | cytoplasmic | APPRDFAAYRS [SEQ ID NO: 23] |
| CD20t | CD28 signal peptide-CD20 | MLRLLLALNLFPSIQVTGNCEPANPSEKNSPST |
| v5.3 | extracellular 166-188-CASPR2 | QYCYSIQSIIGGVIAVVIFTILCTLVFLIRYMFRH |
| | transmembrane-CASPR 2 | KGTYHTNEAKGAESAESADAAIMNNDPNFTE |
| | cytoplasmic | TIDESKKEWLI [SEQ ID NO: 24] |
| CD20t | CD28 signal peptide-CD20 168- | MLRLLLALNLFPSIQVTGEPANPSEKNSPSTQY |
| v5.4 | 188-CASPR2 transmembrane- | CYSIQSIIGGVIAVVIFTICTLVFLIRYLMFRHK |
| | CASPR2 cytoplasmic | GTYHTNEAKGAESAESADAAIMNNDPNFTETI |
| | | DESKKEWLI [SEQ ID NO: 25] |

Applicants also combined CD20's minimal antibody binding epitope with different signal peptides, transmembrane and cytoplasmic domains that are known to promote good surface protein trafficking and expression [15]. Applicants first tested human granulocyte-macrophage colony-stimulating factor receptor (GM-CSFR) signal peptide and epidermal growth factor receptor (EGFR) transmembrane domain, as truncating EGFR's intracellular domain leads to a membrane bound protein [4]. Refer to the constructs designated CD20vt v4 and CD20t v5.1.

Next, Applicants combined different lengths of CD20's antibody binding region with CD28 signal peptide and with transmembrane and cytoplasmic domains derived from CD28 and contactin-associated protein-like 2 (CASPR2) to generate chimeric proteins since constructs incorporating CD28 and CASPR2 transmembrane/cytoplasmic domains lead to membrane bound proteins [16, 17]. Refer to the constructs designated CD20t v5.2, CD20t v5.3 and CD20t v5.4.

With reference to the data presented in FIG. 1, none of the truncated or chimeric CD20 molecules were detected on HEK293 cell membranes, although the CD20 gene was expressed as reflected by the production of green fluorescent protein.

### Example 3: Phosphorylation-Impaired CD20 is Detected on Cell Membrane

Since none of the truncated or chimeric versions of CD20 were detected on HEK293 cell membranes, Applicants altered the approach to produce a membrane bound non-signalling CD20 molecule.

CD20 is heavily phosphorylated on serine and threonine residues in normal and malignant B cells and this process is linked to B cell proliferation [18]. Rituximab binding to CD20 initiates a cascade of signals that may play a role in antibody-mediated cell killing. CD20 is associated with lyn, fyn, Ick and p75/85 kinase [19] and its engagement leads to the activation of PLCy via src-family kinases [20].

Although reports relating to CD20's function are conflicting, it seems clear that phosphorylation and/or association with kinases are important mechanisms for its activation, hence, a CD20 molecule with mutated phosphorylation sites would retain the antibody binding regions while unable to transduce signal.

Most phosphorylation events in eukaryotic cells occur on serine (S), threonine (T) and tyrosine (Y) residues [28]. CD20's cytoplasmic sequences contain 15 serine, 11 threonine and no tyrosine residues (FIG. 2). CD20 therefore has 26 possible phosphorylation sites, however there is only direct evidence of two [21]. To reduce the number of modifications introduced, thus reducing the alterations in protein structure, Applicants employed three different predictors to select potential phosphorylation sites for modification (i) NetPhos3.1, which predicts S, T or Y phosphorylation sites in eukaryotic proteins using ensembles of neural networks [22]; (ii) the published crystal structure of the CD20 and Rituximab complex [21]; and (iii) sequence identity between human and mouse CD20, based on the hypothesis that important functional residues will be conserved (BLASTp).

The combination of these methods led to the identification of putative phosphorylation sites in cytoplasmic regions 1 and 3 (FIG. 2).

Ten amino acids phosphorylated in the crystal structure and/or identified by both NetPhos prediction and mouse sequence were substituted by alanine (A) as it eliminates the amino acid side chains but does not alter the main-chain conformation nor imposes major electrostatic or steric changes [29].

CD20 variants comprising these phospho-mutations and conservative truncations designed by Julie Deans et. al. to study intermolecular interactions with CD20 [19] were generated. Refer to Table 2 as follows:

TABLE 2
Combination of CD20 targeted mutations and truncations

| CD20 | | |
|---|---|---|
| version | Description | Sequence (mutations in bold) |
| C1C252 | Cytoplasmic region 1: | MAAPRNAVNGAFPAEPMKGPIAMQSGPKPLFRRMAA |
| | T2A, T3A, S7A, T11A, | LVGPTQSFFMRESKALGAVQIMNGLFHIALGGLLMIPA |
| | S35A, S36A, T51A | GIYAPICVTVWYPLWGGIMYIISGSLLAATEKNSRKCL |
| | C-terminus: deletion | VKGKMIMNSLSLFAAISGMILSIMDILNIKISHFLKMES |
| | from S253 [19] | LNFIRAHTPYINIYNCEPANPSEKNSPSTQYCYSIQSLF |
| | | LGILSVMLIFAFFQELVIAGIVENEWKRTCSRPKSNIVL |
| | | LSAEEKKEQTIEIKEEVVGLTET [SEQ ID NO: 5] |
| N51C3 | N-terminus: CΔ50 [19] | MTLGAVQIMNGLFHIALGGLLMIPAGIYAPICVTVWYP |
| | Cytoplasmic region 3: | LWGGIMYIISGSLLAATEKNSRKCLVKGKMIMNSLSLF |
| | S225A, S231A, T239A | AAISGMILSIMDILNIKISHFLKMESLNFIRAHTPYINIY |
| | | NCEPANPSEKNSPSTQYCYSIQSLFLGILSVMLIFAFFQ |
| | | ELVIAGIVENEWKRTCSRPKANIVLLAAEEKKEQAIEI |
| | | KEEVVGLTETSSQPKNEEDIEIIPIQEEEEEETETNFPEP |
| | | PQDQESSPIENDSSP [SEQ ID NO: 6] |
| C1C3 | Cytoplasmic region 1: | MAAPRNAVNGAFPAEPMKGPIAMQSGPKPLFRRMAA |
| | T2A, T3A, S7A, T11A, | LVGPTQSFFMRESKALGAVQIMNGLFHIALGGLLMIPA |
| | S35A, S36A, T51A | GIYAPICVTVWYPLWGGIMYIISGSLLAATEKNSRKCL |
| | Cytoplasmic region 3: | VKGKMIMNSLSLFAAISGMILSIMDILNIKISHFLKMES |
| | S225A, S231A, T239A | LNFIRAHTPYINIYNCEPANPSEKNSPSTQYCYSIQSLF |
| | | LGILSVMLIFAFFQELVIAGIVENEWKRTCSRPKANIVL |
| | | LAAEEKKEQAIEIKEEVVGLTETSSQPKNEEDIEIIPIQ |
| | | EEEEEETETNFPEPPQDQESSPIENDSSP [SEQ ID |
| | | NO: 7] |
| N51 | N-terminus: CΔ50 [19] | MTLGAVQIMNGLFHIALGGLLMIPAGIYAPICVTVWYP |
| | | LWGGIMYIISGSLLAATEKNSRKCLVKGKMIMNSLSLF |
| | | AAISGMILSIMDILNIKISHFLKMESLNFIRAHTPYINIY |
| | | NCEPANPSEKNSPSTQYCYSIQSLFLGILSVMLIFAFFQ |
| | | ELVIAGIVENEWKRTCSRPKSNIVLLSAEEKKEQTIEIK |
| | | EEVVGLTETSSQPKNEEDIEIIPIQEEEEEETETNFPEPP |
| | | QDQESSPIENDSSP [SEQ ID NO: 8] |
| C252 | C-terminus: deletion | MTTPRNSVNGTFPAEPMKGPIAMQSGPKPLFRRMSSL |
| | from S253 [19] | VGPTQSFFMRESKTLGAVQIMNGLFHIALGGLLMIPAG |
| | | IYAPICVTVWYPLWGGIMYIISGSLLAATEKNSRKCLV |
| | | KGKMIMNSLSLFAAISGMILSIMDILNIKISHFLKMESL |
| | | NFIRAHTPYINIYNCEPANPSEKNSPSTQYCYSIQSLFL |
| | | GILSVMLIFAFFQELVIAGIVENEWKRTCSRPKSNIVLL |
| | | SAEEKKEQTIEIKEEVVGLTET [SEQ ID NO: 9] |
| N51C252 | N-terminus: CA50 [19] | MTLGAVQIMNGLFHIALGGLLMIPAGIYAPICVTVWYP |
| | C-terminus: deletion | LWGGIMYIISGSLLAATEKNSRKCLVKGKMIMNSLSLF |
| | from $253 [19] | AAISGMILSIMDILNIKISHFLKMESLNFIRAHTPYINIY |
| | | NCEPANPSEKNSPSTQYCYSIQSLFLGILSVMLIFAFFQ |
| | | ELVIAGIVENEWKRTCSRPKSNIVLLSAEEKKEQTIEIK |
| | | EEVVGLTET [SEQ ID NO: 10] |

HEK293 cells were transfected with the mutated/truncated versions of CD20 included in a LeGO plasmid backbone. All combinations of phospho-mutated and conservatively truncated CD20 versions were detected on HEK293 cell membrane, at different levels of expression. Next, Applicants transduced primary T cells using Lentivirus containing phospho-mutated/truncated CD20 transgenes (FIG. 3).

While all truncated/mutated CD20 molecules were detected on HEK293 cell membrane (FIG. 3), C₂₅₂ (SEQ ID NO: 9) and N₅₁ (SEQ ID NO: 8) proteins were not detected on the surface of primary T cells and the truncation of both cytoplasmic chains resulted in low level expression of N₅₁C₂₅₂ (SEQ ID NO: 10). Surprisingly, when combining the single truncations with mutations in C₁ and C₃ respectively, membrane detection was restored (FIG. 3).

As cells producing C₁C₃ (SEQ ID NO: 7) and C₁-C₂₅₂ proteins have similar membrane levels as CD20WT, further studies focused on evaluating those two CD20 variants.

### Example 4: Mutations to Predicted Phosphorylation Sites Permit Rituximab Mediated

CDC in CD20 mutant transduced HG3 cells in vitro CD20KO HG3 cells transduced with LeGO_CD20WT, LeGO_C₁C₃ or LeGO_C₁C₂₅₂ and a sham transduced control were incubated with Rituximab and CDC was induced using baby rabbit complement. Rituximab mediated a significant cell cytotoxicity specific to CD20 (FIG. 4A) that was not affected by mutations in predicted phosphorylation sites or truncating cytoplasmic domain 3. The differences in transduction efficiency (FIG. 4B) for each mutant and CD20WT account for the differences in cytotoxicity and the <100% cytotoxicity observed.

### Example 5: Rituximab Surrogate Specifically Depletes hCD20WT+, C₁C₃ and C₁C₂₅₂-T Cells In Vivo

hCD20WT-GFP, C₁C₃-GFP, C₁C₂₅₂-GFP mouse T cells where adoptively transferred to mice. Administration of anti-CD20 antibody 2H7, a Rituximab surrogate adapted for use in mice, mediated significant reduction (~65%) in the hCD20+ cell population of all mice when compared with mice given the control antibody. Importantly, the depleted fraction consists of the highest transgene expressing cells, and there was no difference in the reduction of C₁C₃-GFP and C₁C₂₅₂-GFP cells compared to hCD20WT-GFP cells, indicating that intracellular mutations and/or truncation in CD20 did not affect 2H7-mediated killing.

### Example 6: C₁C₃, and C₁C₂₅₂ are Detected on the Cell Surface by Various Anti-CD20 Antibodies

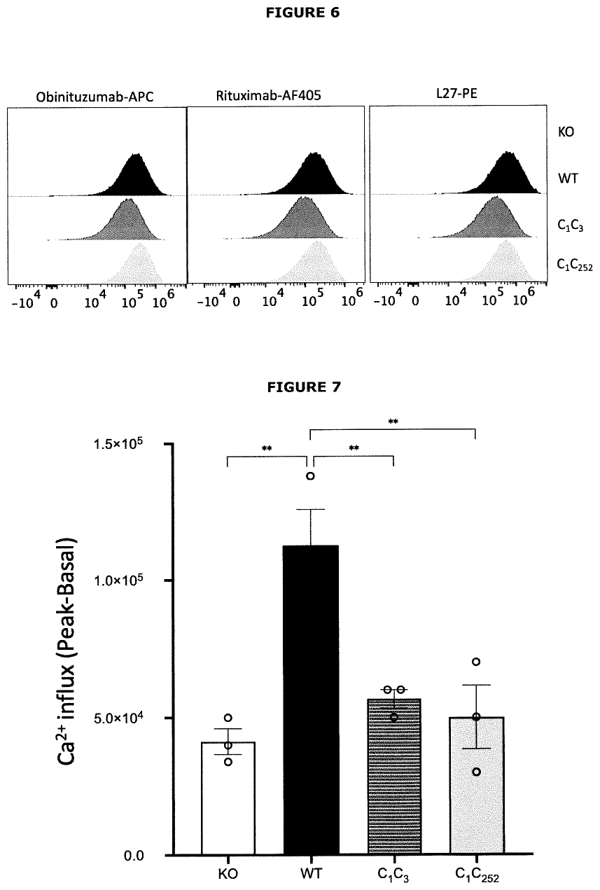

The FDA recommends that long-term follow-up studies for integrating vectors should last for 15 years [30]. Applicants propose that their novel CD20 protein can be used not only as a cell therapies safety switch but also as a selection marker for long term detection of genetically modified cells. Applicants tested the binding of three anti-CD20 antibodies to CD20WT, C₁C₃, and C₁C₂₅₂ sorted CD20+ transduced HG3 cells (FIG. 6). Obinituzumab-APC, Rituximab-AF405 and L27-PE all detect CD20WT and mutants. More importantly, the clinically relevant L27-PE, which is used in hospital clinical laboratories to detect CD20+ cells can be used also to detect cell therapies that include mutant CD20.

### Example 7: Mutation of Phosphorylation Sites Impairs Ca²⁺ Influx Induced by CD20 Engagement

CD20 hyper-crosslinking, or crosslinking of CD20-bound Rituximab, stimulates mobilisation of CD20 into lipid rafts with an increased Ca²⁺ influx [31]. Across many cell types, Ca²⁺ influx promotes cell activation, proliferation and/or differentiation [32], and may be undesirable when seeking to deplete a gene-modified cell population. Ca²⁺ influx induced by hyper-crosslinking of CD20WT, C₁C₃ and C₁C₂₅₂ or in the absence of CD20 was evaluated in the CD20KO HG3 B cell line (FIG. 7). CD20 mutants induce significantly lower levels of Ca²⁺ influx than CD20WT (P≤0.01) and comparable levels to the untransduced CD20KO cells, suggesting that mutation of intracellular CD20 phosphorylation sites can prevent Ca²⁺ influx, and its downstream impacts, upon CD20 crosslinking.

### Example 8: B Cell Phosphorylation Status is Impacted by Activation of C₁C₃

Phospho-kinase activity is a major functional read-out for signalling proteins. Akt, p38MAPK and ERK are among the shared kinases that play a crucial role in the signalling pathways involved in T and B cell activation [33, 34]. CD20 is a key signalling protein that regulates B cell activation [35] and is differentially phosphorylated in resting and activated cells. We evaluated the impact of mutations in CD20's predicted phosphorylation sites in the overall phosphorylation status of downstream kinases in B cells after CD20 engagement. Rituximab-induced activation of C₁C₃ leads to a phosphorylation state comparable to that of CD20 deficient B cells (FIG. 8), indicating that CD20 regulation of B cell activation can be reduced by impairing CD20 phosphorylation. Phosphorylation of cytoplasmic domain 3 appears to be of high relevance as C₁C₂₅₂ shows a phosphorylation pattern comparable to CD20WT, despite the mutations of phosphorylation sites in cytoplasmic domain 1. Notably, phosphorylation of ERK1/2 leads to activation of transcription factors involved in cell proliferation and survival in B and T cells. While C₁C₂₅₂ induces phosphorylation of these kinases comparable to CD20WT, activation of C₁C₃ leads to approximately 3 times less phosphorylation of ERK1/2, which can result in less activation not only of B, but also of T cells.

### Example 9: Therapeutic Utility of Modified CD20 Proteins

A transgene encoding a modified CD20 protein according to the present invention can potentially be incorporated into gene modified (a) T-cells, including CAR T-cells [36], T-cells expressing a transgenic T-cell receptor (TCR) [37], or T-cells expressing another transgenic or synthetic protein [38]; (b) natural killer (NK) cells [39]; (c) B-cells [40]; (d) myeloid cells [41], including myeloid cells modified to express transgenic receptors, such as a CAR, TCR, B-cell receptor (BCR) or NK receptor, or expressing another transgenic or synthetic protein; (e) cell lines, including induced pluripotent stem cell (iPSC)-derived cell lines [42], and cell lines of haematopoietic origin, where these cell lines have been modified to express a transgenic or synthetic protein; and (f) haematopoietic stem cells [43] modified to express a transgenic or synthetic protein, or edited to correct an inherited defect, where the transgenic or synthetic protein provides a therapeutic benefit.

In addition, nucleic acid molecules encoding the modified CD20 proteins described herein may be included in an RNA or DNA product intended to elicit transient transfection or long-term transduction of stem cells, non-haematopoietic stem cells and immune cells, or in a viral vector intended to elicit transient or long-term expression of a transgene, both within a human recipient.

In each case, the modified CD20 may serve as (i) a tag to identify, select, or purify gene-transduced cells during and after the cellular product manufacturing process; (ii) a tag to allow identification and/or characterisation of gene-transduced or gene-transfected cells within patient-derived samples or biopsies, including by flow cytometry or immunohistochemistry; and/or (iii) as a safety switch to enable rapid depletion of gene-transduced cells using a therapy directed against CD20, such as Rituximab, Obinutuzumab, or Ocrelizumab.

Modified CD20 proteins that incorporate only changes to intracellular phosphorylation sites retain the native transmembrane and extracellular CD20 sequence. In comparison to modified CD20 proteins that incorporate multiple extracellular mutations, this reduces the risk of antibody-mediated immunogenicity against gene-transduced or gene-transfected cells.

### Example 10: Extracellular Mutations for Exclusive Binding of Rituximab or Obinutuzumab

Circulating levels of Rituximab or Obinutuzumab might limit the use of CD20 as a safety switch in B-cell malignancies patients treated with those antibodies prior to gene therapy. Here, Applicants targeted mutations to the second extracellular loop of CD20 to allow for exclusive binding of Rituximab or Obinutuzumab.

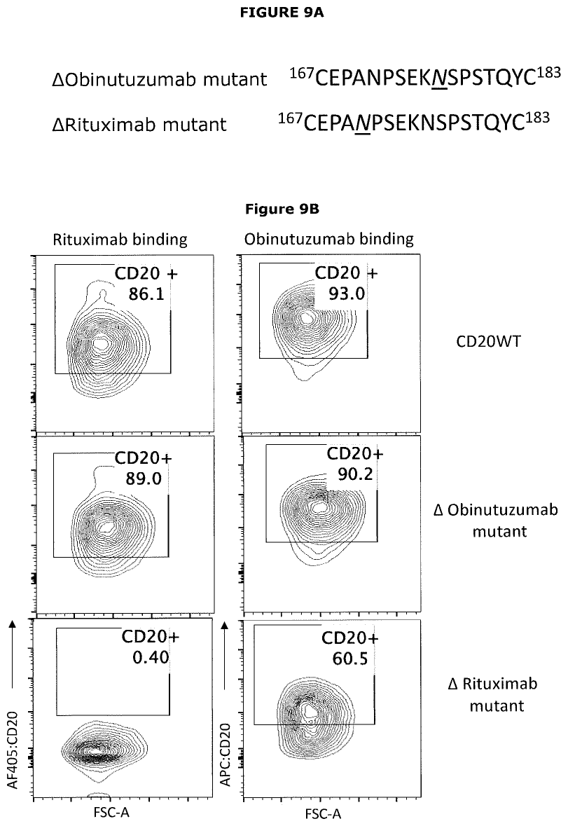

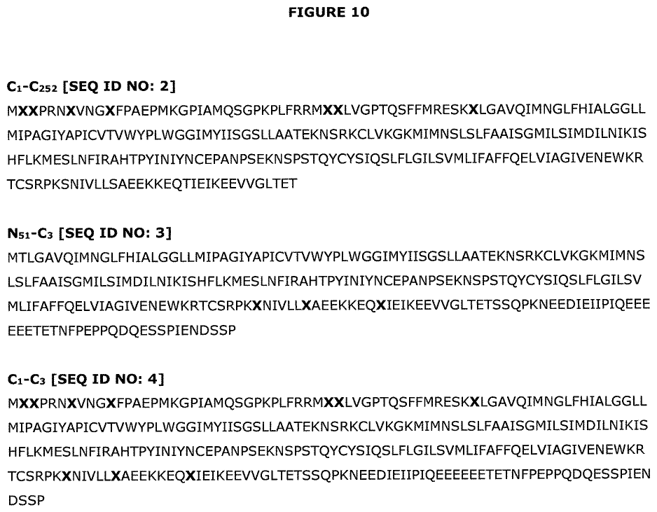

In 2013 Klein et. al. reviewed the epitope interactions of monoclonal antibodies targeting CD20 [8](FIG. 9).

Asparagine 171 is essential for Rituximab binding to CD20, however, Obinutuzumab binding is maintained when this amino acid is exchanged by almost any other amino acid. Mutation of N176 abrogates Obinutuzumab binding but maintain Rituximab binding. Conservative substitutions of those asparagine residues to aspartic acid (i.e. N171D or N176D, underlined in FIG. 9A can lead to a CD20 version that exclusively binds Obinutuzumab or Rituximab respectively. Applicants designed a ΔObinutuzumab mutant (CD20 N176D and a ΔRituximab mutant (CD20 N171D). The ΔObinutuzumab mutant retained binding of both Rituximab and Obinutuzumab, however, and more relevant due to the wide spread use pf Rituximab, the ΔRituximab mutant does not allow for binding of Rituximab but retains binding of Obinutuzumab. A CD20 molecule combining this extracellular point mutation with the intracellular mutations (C₁C₃) can be used in patients previously treated with Rituximab that are prescribed CAR-T cell therapy containing a safety switch. The detection by anti-CD20 antibodies and CD20 mediated killing of this protein (ΔRituximab-C₁C₃) will be tested in further studies.

### Example 11: Conclusions

The tertiary structure of CD20 is not maintained by major disruptions in its secondary structure, however, minimal changes can lead to successful production of a protein that is detected on the cell surface. Applicants have designed putative CD20 phospho-mutants that can be detected in the cell membrane of a HEK293 (human embryonic kidney) cell line, HG3 (B cells) and most importantly in primary T cells. These phospho-mutants impair the CD20 signalling cascade in B cells, exemplified by impairment in Ca²⁺ influx and overall kinase phosphorylation, while permitting both detection by CD20 antibodies, and antibody dependent cytotoxicity in B cells in vitro and T cells in vivo. By mutating key residues among CD20's predicted phosphorylation sites, Applicants were able to retain cell surface expression, detection and cell death induced by clinically relevant antibodies while abrogating CD20's signalling functions. This minimises the potential risk of cell activation caused by engagement of the safety switch. While Ca²⁺ influx was abrogated by both CD20 mutants, only C₁C₃ had an impact in the phosphorylation status of B cells, highlighting the relevance of phosphorylation sites in cytoplasmic domain 3.

Accordingly, the CD20 phospho-mutants described herein are compelling candidates for development of a safety switch and detection tool or as a selection marker for gene-modified cellular therapies.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Gene therapy needs a long-term approach. Nature Medicine, 2021. 27(4): p. 563-563.
2. Goswami, R., et al., Gene Therapy Leaves a Vicious Cycle. Frontiers in Oncology, 2019. 9.
3. Neelapu, S. S., et al., Chimeric antigen receptor T-cell therapy—assessment and management of toxicities. Nat Rev Clin Oncol, 2018. 15(1): p. 47-62.
4. Wang, X., et al., A transgene-encoded cell surface polypeptide for selection, in vivo tracking, and ablation of engineered cells. Blood, 2011. 118(5): p. 1255-63.
5. Moghanloo, E., et al., Remote controlling of CAR-T cells and toxicity management: Molecular switches and next generation CARs. Translational Oncology, 2021. 14(6): p. 101070.
6. Services., U.S.D.o.H.a.H., Long Term Follow-Up After Administration of Human Gene Therapy Products, F.a.D.A.C.f.B.E.a. Research, Editor. 2020.
7. Agency, E. M., Guideline on quality, non-clinical and clinical aspects of medicinal products containing genetically modified cells. 2020.
8. Klein, C., et al., Epitope interactions of monoclonal antibodies targeting CD20 and their relationship to functional properties. MAbs, 2013. 5(1): p. 22-33.
9. Gabriela, P. and M. Marek, The regulation and function of CD20: an “enigma” of B-cell biology and targeted therapy. Haematologica, 2020. 105(6): p. 1494-1506.
10. Uchida, J., et al., Mouse CD20 expression and function. Int Immunol, 2004. 16(1): p. 119-29.
11. Li, H., et al., Store-operated cation entry mediated by CD20 in membrane rafts. J Biol Chem, 2003. 278(43): p. 42427-34.
12. Steele, L., et al., Non-redundant activity of GSK-3a and GSK-3Q in T cell-mediated tumor rejection. iScience, 2021. 24(6): p. 102555.
13. Mathas, S., et al., Anti-CD20- and B-cell Receptor-mediated Apoptosis: Evidence for Shared Intracellular Signaling Pathways1. Cancer Research, 2000. 60(24): p. 7170-7176.
14. Deans, J. P., H. Li, and M. J. Polyak, CD20-mediated apoptosis: signalling through lipid rafts. Immunology, 2002. 107(2): p. 176-182.
15. Philip, B., et al., A highly compact epitope-based marker/suicide gene for easier and safer T-cell therapy. Blood, 2014. 124(8): p. 1277-1287.
16. Comoletti, D. 2021.
17. Weng, J., et al., A novel generation 1928zT2 CAR T cells induce remission in extramedullary relapse of acute lymphoblastic leukemia. J Hematol Oncol, 2018. 11(1): p. 25.
18. Tedder, T. F. and S. F. Schlossman, Phosphorylation of the B1 (CD20) molecule by normal and malignant human B lymphocytes. Journal of Biological Chemistry, 1988. 263(20): p. 10009-10015.
19. Deans, J. P., et al., Association of75/80-kDa Phosphoproteins and the Tyrosine Kinases Lyn, Fyn, and Lck with the B Cell Molecule CD20: EVIDENCE AGAINST INVOLVEMENT OF THE CYTOPLASMIC REGIONS OF CD20 (*). Journal of Biological Chemistry, 1995. 270(38): p. 22632-22638.
20. Shan, D., J. A. Ledbetter, and O. W. Press, Signaling events involved in anti-CD20-induced apoptosis of malignant human B cells. Cancer Immunol Immunother, 2000. 48(12): p. 673-83.
21. Rougé, L., et al., Structure of CD20 in complex with the therapeutic monoclonal antibody rituximab. Science, 2020. 367(6483): p. 1224-1230.
22. Blom, N., S. Gammeltoft, and S. Brunak, Sequence and structure-based prediction of eukaryotic protein phosphorylation sites. J Mol Biol, 1999. 294(5): p. 1351-62.
23. Astronomo, R. D. and D. R. Burton, Carbohydrate vaccines: developing sweet solutions to sticky situations? Nature Reviews Drug Discovery, 2010. 9(4): p. 308-324.
24. Nishikawa, M. and L. Huang, Nonviral vectors in the new millennium: delivery barriers in gene transfer. Hum Gene Ther, 2001. 12(8): p. 861-70.
25. Schenborn, E. T. and R. C. Mierendorf, Jr., A novel transcription property of SP6 and T7 RNA polymerases: dependence on template structure. Nucleic Acids Res, 1985. 13(17): p. 6223-36.
26. Nacheva, G. A. and A. Berzal-Herranz, Preventing nondesired RNA-primed RNA extension catalyzed by T7 RNA polymerase. Eur J Biochem, 2003. 270(7): p. 1458-65.
27. George, P., et al., Third-generation anti-CD19 chimeric antigen receptor T-cells incorporating a TLR2 domain for relapsed or refractory B-cell lymphoma: a phase I clinical trial protocol (ENABLE). BMJ Open, 2020. 10(2): p. e034629.
28. Ardito, F., et al., The crucial role of protein phosphorylation in cell signaling and its use as targeted therapy (Review). Int J Mol Med, 2017. 40(2): p. 271-280.
29. Lefevre, F., M.-H. Remy, and J.-M. Masson, Alanine-stretch scanning mutagenesis: a simple and efficient method to probe protein structure and function. Nucleic Acids Research, 1997. 25(2): p. 447-448.
30. Sciences, N.A.o., The National Academies Collection: Reports funded by National Institutes of Health, in Exploring Novel Clinical Trial Designs for Gene-Based Therapies: Proceedings of a Workshop. 2020, National Academies Press (US).
31. Janas, E., et al., Rituxan (anti-CD20 antibody)-induced translocation of CD20 into lipid rafts is crucial for calcium influx and apoptosis. Clin Exp Immunol, 2005. 139(3): p. 439-46.
32. Trebak, M. and J. P. Kinet, Calcium signalling in T cells. Nat Rev Immunol, 2019. 19(3): p. 154-169.
33. Hwang, J. R., et al., Recent insights of T cell receptor-mediated signaling pathways for T cell activation and development. Exp Mol Med, 2020. 52(5): p. 750-761.
34. Toapanta, F., P. Bernal, and M. Sztein, Diverse phosphorylation patterns of B cell receptor-associated signaling in naïve and memory human B cells revealed by phosphoflow, a powerful technique to study signaling at the single cell level. Frontiers in Cellular and Infection Microbiology, 2012. 2.
35. Valentine, M. A., et al., Phosphorylation of the CD20 phosphoprotein in resting B lymphocytes. Regulation by protein kinase C. J Biol Chem, 1989. 264(19): p. 11282-7.
36. Li, D., et al., Genetically engineered T cells for cancer immunotherapy. Signal Transduction and Targeted Therapy, 2019. 4(1): p. 35.
37. Tsimberidou, A.-M., et al., T-cell receptor-based therapy: an innovative therapeutic approach for solid tumors. Journal of Hematology & Oncology, 2021. 14(1): p. 102.
38. Johansen, K. H., How CRISPR/Cas9 Gene Editing Is Revolutionizing T Cell Research. DNA and Cell Biology, 2021. 41(1): p. 53-57.
39. Liu, S., et al., NK cell-based cancer immunotherapy: from basic biology to clinical development. Journal of Hematology & Oncology, 2021. 14(1): p. 7.
40. Ueda, N., et al., Immunotherapy perspectives in the new era of B-cell editing. Blood Advances, 2021. 5(6): p. 1770-1779.
41. Kaczanowska, S., et al., Genetically engineered myeloid cells rebalance the core immune suppression program in metastasis. Cell, 2021. 184(8): p. 2033-2052.e21.
42. Schambach, A., et al., Generation and genetic modification of induced pluripotent stem cells. Expert Opin Biol Ther, 2010. 10(7): p. 1089-103.
43. Naldini, L., Genetic engineering of hematopoiesis: current stage of clinical translation and future perspectives. EMBO Mol Med, 2019. 11(3).
44. Milone et al, Mol. Ther. 17(8): 1453.
45. https://www.ema.europa.eu/en/documents/scientific-guideline/guideline-quality-non-clinical-clinical-aspects-medicinal-products-containing-genetically-modified_en-0.pdf.
46. iScience 2021; PubMed ID 33615197.
47. Nishikawa, et al. Hum Gene Ther., 12(8):861-70 (2001).
48. U.S. Pat. No. 5,350,674.
49. U.S. Pat. No. 5,585,362.
50. Ghosh et al., 1991 Glycobiology 5: 505-10.
51. Schenbom and Mierendorf, Nuc Acids Res., 13:6223-36 (1985).
52. Nacheva and Berzal-Herranz, Eur. J. Biochem., 270:1485-65 (2003).
53. US 2004/0014645, US 2005/0052630A1, US 2005/0070841A1, US 2004/0059285A1, US 2004/0092907A1.
54. U.S. Pat. Nos. 6,678,556, 7,171,264, and 7,173,116.
55. U.S. Pat. Nos. 6,567,694; 6,516,223, 5,993,434, 6,181,964, 6,241,701, and 6,233,482.
56. US20070128708A1.
57. Cougot, et al., Trends in Biochem. Sci., 29:436-444 (2001).
58. https://www.swisssidechain.ch/browse.php.
59. Ramanathan et al. (2016) Nucleic Acid Res. 44(16):7511-7526.
60. Walshe et al. (2008) J Biol Chem 283(25):16971-84.
61. Franke et al. (2011) PLoS One 6(2):e16596.

1. A human CD20 protein comprising:
(i) at least one mutation causing a truncation to any one or more of amino acid residues 1-56 set forth in SEQ ID NO: 1;
(ii) at least one mutation causing a truncation to any one or more of amino acid residues 210-297 set forth in SEQ ID NO: 1;
(iii) at least one mutation to a threonine or a serine located within amino acid residues 1-56 set forth in SEQ ID NO: 1;
(iv) at least one mutation to a threonine or a serine located within amino acid residues 210-297 set forth in SEQ ID NO: 1; or
(v) a combination comprising any one of (i) to (iv)
wherein any one of (i) to (v) causes reduced or abrogated intracellular signalling when the modified CD20 protein is attached to or associated with the membrane of a cell.

2. The human CD20 protein according to claim 1 comprising:
(i) at least one mutation causing a truncation to any one or more of amino acid residues 1-56 set forth in SEQ ID NO: 1; and
(ii) at least one mutation to a threonine or a serine located within amino acid residues 210-297 set forth in SEQ ID NO: 1.

3. The human CD20 protein according to claim 1 comprising:
(i) at least one mutation to a threonine or a serine located within amino acid residues 1-56 set forth in SEQ ID NO: 1; and
(ii) at least one mutation causing a truncation to any one or more of amino acid residues 210-297 set forth in SEQ ID NO: 1.

4. The human CD20 protein according to claim 1 comprising:
(i) at least one mutation to a threonine or a serine located within amino acid residues 1-56 set forth in SEQ ID NO: 1; and
(ii) at least one mutation to a threonine or a serine located within amino acid residues 210-297 set forth in SEQ ID NO: 1.

5. The human CD20 protein according to claim 1 or claim 2, wherein amino acid residues 1-50 set forth in SEQ ID NO: 1 are truncated.

6. The human CD20 protein according to claim 1 or claim 3, wherein amino acid residues 253-297 set forth in SEQ ID NO: 1 are truncated.

7. The human CD20 protein according to claim 1, claim 3 or claim 4 wherein the at least one mutation to a threonine or a serine located within amino acid residues 1-56 set forth in SEQ ID NO: 1 comprises a mutation to any one or more of T2, T3, S7, T11, S35, S36 and T51.

8. The human CD20 protein according to claim 1, claim 2 or claim 4 wherein the at least one mutation to a threonine or a serine located within amino acid residues 210-297 set forth in SEQ ID NO: 1 comprises a mutation to any one or more of S225, S231 and T239.

9. The human CD20 protein according to any one of claims 1 to 8 wherein the at least one mutation is selected from a substitution mutation, a deletion mutation and an insertion mutation.

10. The human CD20 protein according to claim 9 wherein the substitution mutation comprises substitution with at least one naturally or non-naturally occurring amino acid residue.

11. The human CD20 protein according to claim 7 wherein the mutation is selected from any one or more of T2A, T3A, S7A, T11A, S35A, S36A and T51A.

12. The human CD20 protein according to claim 8 wherein the mutation is selected from any one or more of S225A, S231A and T239A.

13. The human CD20 protein according to claim 1 comprising or consisting in the sequence set forth in any one of SEQ ID NO:1, SEQ ID NO: 2, SEQ ID NO: 3, SEQ ID NO: 4, SEQ ID NO: 5, SEQ ID NO: 6 and SEQ ID NO: 7, or a sequence comprising at least 80%, 85%, 86%, 87%, 88%, 89%, 90%, 91%, 92%, 93%, 94%, 95%, 96%, 97%, 98% and 99% sequence identity to SEQ ID NO: 2, SEQ ID NO: 3, SEQ ID NO: 4, SEQ ID NO: 5, SEQ ID NO: 6 or SEQ ID NO: 7, respectively.

14. The human CD20 protein according to any one of claims 1 to 13, further comprising at least one mutation to the extracellular domain defined by amino acid residues 142-188, preferably amino acid residues 167-183, as set forth in SEQ ID NO: 1, which mutation eliminates binding by Rituximab or Obinutuzumab or Ocrelizumab.

15. The human CD20 protein according to claim 14 wherein the at least one mutation comprises a mutation to amino acid residues N171, S173 and/or N176 set forth in SEQ ID NO: 1.

16. A cell expressing the human CD20 protein according to any one of claims 1 to 15.

17. The cell according to claim 16 which comprises a T-cell, a natural killer cell, a B-cell, a myeloid cell, a pluripotent stem cell, a haematopoietic stem cell, a non-haematopoietic stem cell and a human cell.

18. A nucleic acid comprising sequence encoding the human CD20 protein according to any one of claims 1 to 15.

19. A vector comprising the nucleic acid according to claim 18.

20. The vector according to claim 19 which is a viral vector.


SEQUENCE LISTING (169 sequences)

SEQ ID NO: 1 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 2 - protein, 252 aa, Homo sapiens
MXXPRNXVNGXFPAEPMKGPIAMQSGPKPLFRRMXXLVGPTQSFFMRESKXLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTET

SEQ ID NO: 3 - protein, 248 aa, Homo sapiens
MTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKXNIVLLXAEEKKEQXIEIKEEVVGLTETSSQPKNEEDIEIIPIQEEEEEETETNFPEPPQDQESSPIENDSSP

SEQ ID NO: 4 - protein, 297 aa, Homo sapiens
MXXPRNXVNGXFPAEPMKGPIAMQSGPKPLFRRMXXLVGPTQSFFMRESKXLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKXNIVLLXAEEKKEQXIEIKEEVVGLTETSSQPKNEEDIEIIPIQEEEEEETETNFPEPPQDQESSPIENDSSP

SEQ ID NO: 5 - protein, 252 aa, Homo sapiens
MAAPRNAVNGAFPAEPMKGPIAMQSGPKPLFRRMAALVGPTQSFFMRESKALGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTET

SEQ ID NO: 6 - protein, 248 aa, Homo sapiens
MTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKANIVLLAAEEKKEQAIEIKEEVVGLTETSSQPKNEEDIEIIPIQEEEEEETETNFPEPPQDQESSPIENDSSP

SEQ ID NO: 7 - protein, 297 aa, Homo sapiens
MAAPRNAVNGAFPAEPMKGPIAMQSGPKPLFRRMAALVGPTQSFFMRESKALGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKANIVLLAAEEKKEQAIEIKEEVVGLTETSSQPKNEEDIEIIPIQEEEEEETETNFPEPPQDQESSPIENDSSP

SEQ ID NO: 8 - protein, 248 aa, Homo sapiens
MTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 9 - protein, 252 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTET

SEQ ID NO: 10 - protein, 203 aa, Homo sapiens
MTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTET

SEQ ID NO: 11 - protein, 56 aa, Homo sapiens
MTTPRNSVNGTFPAEPMKGPIAMQSGPKPLFRRMSSLVGPTQSFFMRESKTLGAVQ

SEQ ID NO: 12 - protein, 88 aa, Homo sapiens
GIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 13 - protein, 17 aa, Homo sapiens
CEPANPSEKNSPSTQYC

SEQ ID NO: 14 - protein, 188 aa, Homo sapiens
MRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSSSQPKNEEDIEIIPIQEEEEEETETNFPEPPQDQESSPIENDSSP

SEQ ID NO: 15 - protein, 168 aa, Homo sapiens
MNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKS

SEQ ID NO: 16 - protein, 140 aa, Homo sapiens
MKGKMIMNSLSLFAAISGMILSIMDILNIKISHFLKMESLNFIRAHTPYINIYNCEPANPSEKNSPSTQYCYSIQSLFLGILSVMLIFAFFQELVIASQPKNEEDIEIIPIQEEEEEETETNFPEPPQDQESSPIENDSP

SEQ ID NO: 17 - protein, 179 aa, Homo sapiens
MRESKTLGAVQMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKS

SEQ ID NO: 18 - protein, 149 aa, Homo sapiens
MKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIASQPKNEEDIEIIPIQEEEEEETETNFPEPPQDQESSPIENDSSP

SEQ ID NO: 19 - protein, 234 aa, Homo sapiens
MTTPRNSVNGTFPAEPMKGPIAMQSGPKPLFRRMSSLVGPTQSFFMRESKTLGAVQIMNGLFHIALGGLLMIPAGIYAKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 20 - protein, 233 aa, Homo sapiens
MTTPRNSVNGTFPAEPMKGPIAMQSGPKPLFRRMSSLVGPTQSFFMRESKTLGAVQSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 21 - protein, 70 aa, Homo sapiens
MLLLVTSLLLCELPHPAFLLIPNIYNCEPANPSEKNSPSTQYCYSIQIATGMVGALLLLLVVALGIGLFM

SEQ ID NO: 22 - protein, 66 aa, Homo sapiens
MLLLVTSLLLCELPHPAFLLIPCEPANPSEKNSPSTQYCYSIQIATGMVGALLLLLVVALGIGLFM

SEQ ID NO: 23 - protein, 108 aa, Homo sapiens
MLRLLLALNLFPSIQVTGCEPANPSEKNSPSTQYCYSIQSFWVLVVVGGVLACYSLLVTVAFIIFWVRSKRSRLLHSDYMNMTPRRPGPTRKHYQPYAPPRDFAAYRS

SEQ ID NO: 24 - protein, 110 aa, Homo sapiens
MLRLLLALNLFPSIQVTGNCEPANPSEKNSPSTQYCYSIQSIIGGVIAVVIFTILCTLVFLIRYMFRHKGTYHTNEAKGAESAESADAAIMNNDPNFTETIDESKKEWLI

SEQ ID NO: 25 - protein, 108 aa, Homo sapiens
MLRLLLALNLFPSIQVTGEPANPSEKNSPSTQYCYSIQSIIGGVIAVVIFTICTLVFLIRYLMFRHKGTYHTNEAKGAESAESADAAIMNNDPNFTETIDESKKEWLI

SEQ ID NO: 26-28 - skipped (3 entries)

SEQ ID NO: 29 - protein, 4 aa, Homo sapiens
MTTP

SEQ ID NO: 30 - protein, 5 aa, Homo sapiens
MTTPR

SEQ ID NO: 31 - protein, 6 aa, Homo sapiens
MTTPRN

SEQ ID NO: 32 - protein, 7 aa, Homo sapiens
MTTPRNS

SEQ ID NO: 33 - protein, 8 aa, Homo sapiens
MTTPRNSV

SEQ ID NO: 34 - protein, 9 aa, Homo sapiens
MTTPRNSVN

SEQ ID NO: 35 - protein, 10 aa, Homo sapiens
MTTPRNSVNG

SEQ ID NO: 36 - protein, 11 aa, Homo sapiens
MTTPRNSVNGT

SEQ ID NO: 37 - protein, 12 aa, Homo sapiens
MTTPRNSVNGTF

SEQ ID NO: 38 - protein, 13 aa, Homo sapiens
MTTPRNSVNGTFP

SEQ ID NO: 39 - protein, 14 aa, Homo sapiens
MTTPRNSVNGTFPA

SEQ ID NO: 40 - protein, 15 aa, Homo sapiens
MTTPRNSVNGTFPAE

SEQ ID NO: 41 - protein, 16 aa, Homo sapiens
MTTPRNSVNGTFPAEP

SEQ ID NO: 42 - protein, 17 aa, Homo sapiens
MTTPRNSVNGTFPAEPM

SEQ ID NO: 43 - protein, 18 aa, Homo sapiens
MTTPRNSVNGTFPAEPMK

SEQ ID NO: 44 - protein, 19 aa, Homo sapiens
MTTPRNSVNGTFPAEPMKG

SEQ ID NO: 45 - protein, 20 aa, Homo sapiens
MTTPRNSVNGTFPAEPMKGP

SEQ ID NO: 46 - protein, 21 aa, Homo sapiens
MTTPRNSVNGTFPAEPMKGPI

SEQ ID NO: 47 - protein, 22 aa, Homo sapiens
MTTPRNSVNGTFPAEPMKGPIA

SEQ ID NO: 48 - protein, 23 aa, Homo sapiens
MTTPRNSVNGTFPAEPMKGPIAM

SEQ ID NO: 49 - protein, 24 aa, Homo sapiens
MTTPRNSVNGTFPAEPMKGPIAMQ

SEQ ID NO: 50 - protein, 25 aa, Homo sapiens
MTTPRNSVNGTFPAEPMKGPIAMQS

SEQ ID NO: 51 - protein, 26 aa, Homo sapiens
MTTPRNSVNGTFPAEPMKGPIAMQSG

SEQ ID NO: 52 - protein, 27 aa, Homo sapiens
MTTPRNSVNGTFPAEPMKGPIAMQSGP

SEQ ID NO: 53 - protein, 28 aa, Homo sapiens
MTTPRNSVNGTFPAEPMKGPIAMQSGPK

SEQ ID NO: 54 - protein, 29 aa, Homo sapiens
MTTPRNSVNGTFPAEPMKGPIAMQSGPKP

SEQ ID NO: 55 - protein, 30 aa, Homo sapiens
MTTPRNSVNGTFPAEPMKGPIAMQSGPKPL

SEQ ID NO: 56 - protein, 31 aa, Homo sapiens
MTTPRNSVNGTFPAEPMKGPIAMQSGPKPLF

SEQ ID NO: 57 - protein, 32 aa, Homo sapiens
MTTPRNSVNGTFPAEPMKGPIAMQSGPKPLFR

SEQ ID NO: 58 - protein, 33 aa, Homo sapiens
MTTPRNSVNGTFPAEPMKGPIAMQSGPKPLFRR

SEQ ID NO: 59 - protein, 34 aa, Homo sapiens
MTTPRNSVNGTFPAEPMKGPIAMQSGPKPLFRRM

SEQ ID NO: 60 - protein, 35 aa, Homo sapiens
MTTPRNSVNGTFPAEPMKGPIAMQSGPKPLFRRMS

SEQ ID NO: 61 - protein, 36 aa, Homo sapiens
MTTPRNSVNGTFPAEPMKGPIAMQSGPKPLFRRMSS

SEQ ID NO: 62 - protein, 37 aa, Homo sapiens
MTTPRNSVNGTFPAEPMKGPIAMQSGPKPLFRRMSSL

SEQ ID NO: 63 - protein, 38 aa, Homo sapiens
MTTPRNSVNGTFPAEPMKGPIAMQSGPKPLFRRMSSLV

SEQ ID NO: 64 - protein, 39 aa, Homo sapiens
MTTPRNSVNGTFPAEPMKGPIAMQSGPKPLFRRMSSLVG

SEQ ID NO: 65 - protein, 40 aa, Homo sapiens
MTTPRNSVNGTFPAEPMKGPIAMQSGPKPLFRRMSSLVGP

SEQ ID NO: 66 - protein, 41 aa, Homo sapiens
MTTPRNSVNGTFPAEPMKGPIAMQSGPKPLFRRMSSLVGPT

SEQ ID NO: 67 - protein, 42 aa, Homo sapiens
MTTPRNSVNGTFPAEPMKGPIAMQSGPKPLFRRMSSLVGPTQ

SEQ ID NO: 68 - protein, 43 aa, Homo sapiens
MTTPRNSVNGTFPAEPMKGPIAMQSGPKPLFRRMSSLVGPTQS

SEQ ID NO: 69 - protein, 44 aa, Homo sapiens
MTTPRNSVNGTFPAEPMKGPIAMQSGPKPLFRRMSSLVGPTQSF

SEQ ID NO: 70 - protein, 45 aa, Homo sapiens
MTTPRNSVNGTFPAEPMKGPIAMQSGPKPLFRRMSSLVGPTQSFF

SEQ ID NO: 71 - protein, 46 aa, Homo sapiens
MTTPRNSVNGTFPAEPMKGPIAMQSGPKPLFRRMSSLVGPTQSFFM

SEQ ID NO: 72 - protein, 47 aa, Homo sapiens
MTTPRNSVNGTFPAEPMKGPIAMQSGPKPLFRRMSSLVGPTQSFFMR

SEQ ID NO: 73 - protein, 48 aa, Homo sapiens
MTTPRNSVNGTFPAEPMKGPIAMQSGPKPLFRRMSSLVGPTQSFFMRE

SEQ ID NO: 74 - protein, 49 aa, Homo sapiens
MTTPRNSVNGTFPAEPMKGPIAMQSGPKPLFRRMSSLVGPTQSFFMRES

SEQ ID NO: 75 - protein, 50 aa, Homo sapiens
MTTPRNSVNGTFPAEPMKGPIAMQSGPKPLFRRMSSLVGPTQSFFMRESK

SEQ ID NO: 76 - protein, 51 aa, Homo sapiens
MTTPRNSVNGTFPAEPMKGPIAMQSGPKPLFRRMSSLVGPTQSFFMRESKT

SEQ ID NO: 77 - protein, 52 aa, Homo sapiens
MTTPRNSVNGTFPAEPMKGPIAMQSGPKPLFRRMSSLVGPTQSFFMRESKTL

SEQ ID NO: 78 - protein, 53 aa, Homo sapiens
MTTPRNSVNGTFPAEPMKGPIAMQSGPKPLFRRMSSLVGPTQSFFMRESKTLG

SEQ ID NO: 79 - protein, 54 aa, Homo sapiens
MTTPRNSVNGTFPAEPMKGPIAMQSGPKPLFRRMSSLVGPTQSFFMRESKTLGA

SEQ ID NO: 80 - protein, 55 aa, Homo sapiens
MTTPRNSVNGTFPAEPMKGPIAMQSGPKPLFRRMSSLVGPTQSFFMRESKTLGAV

SEQ ID NO: 81 - protein, 56 aa, Homo sapiens; identical to SEQ ID NO: 11

SEQ ID NO: 82-84 - skipped (3 entries)

SEQ ID NO: 85 - protein, 4 aa, Homo sapiens
DSSP

SEQ ID NO: 86 - protein, 5 aa, Homo sapiens
NDSSP

SEQ ID NO: 87 - protein, 6 aa, Homo sapiens
ENDSSP

SEQ ID NO: 88 - protein, 7 aa, Homo sapiens
IENDSSP

SEQ ID NO: 89 - protein, 8 aa, Homo sapiens
PIENDSSP

SEQ ID NO: 90 - protein, 9 aa, Homo sapiens
SPIENDSSP

SEQ ID NO: 91 - protein, 10 aa, Homo sapiens
SSPIENDSSP

SEQ ID NO: 92 - protein, 11 aa, Homo sapiens
ESSPIENDSSP

SEQ ID NO: 93 - protein, 12 aa, Homo sapiens
QESSPIENDSSP

SEQ ID NO: 94 - protein, 13 aa, Homo sapiens
DQESSPIENDSSP

SEQ ID NO: 95 - protein, 14 aa, Homo sapiens
QDQESSPIENDSSP

SEQ ID NO: 96 - protein, 15 aa, Homo sapiens
PQDQESSPIENDSSP

SEQ ID NO: 97 - protein, 16 aa, Homo sapiens
PPQDQESSPIENDSSP

SEQ ID NO: 98 - protein, 17 aa, Homo sapiens
EPPQDQESSPIENDSSP

SEQ ID NO: 99 - protein, 18 aa, Homo sapiens
PEPPQDQESSPIENDSSP

SEQ ID NO: 100 - protein, 19 aa, Homo sapiens
FPEPPQDQESSPIENDSSP

SEQ ID NO: 101 - protein, 20 aa, Homo sapiens
NFPEPPQDQESSPIENDSSP

SEQ ID NO: 102 - protein, 21 aa, Homo sapiens
TNFPEPPQDQESSPIENDSSP

SEQ ID NO: 103 - protein, 22 aa, Homo sapiens
ETNFPEPPQDQESSPIENDSSP

SEQ ID NO: 104 - protein, 23 aa, Homo sapiens
TETNFPEPPQDQESSPIENDSSP

SEQ ID NO: 105 - protein, 24 aa, Homo sapiens
ETETNFPEPPQDQESSPIENDSSP

SEQ ID NO: 106 - protein, 25 aa, Homo sapiens
EETETNFPEPPQDQESSPIENDSSP

SEQ ID NO: 107 - protein, 26 aa, Homo sapiens
EEETETNFPEPPQDQESSPIENDSSP

SEQ ID NO: 108 - protein, 27 aa, Homo sapiens
EEEETETNFPEPPQDQESSPIENDSSP

SEQ ID NO: 109 - protein, 28 aa, Homo sapiens
EEEEETETNFPEPPQDQESSPIENDSSP

SEQ ID NO: 110 - skipped

SEQ ID NO: 111 - protein, 30 aa, Homo sapiens
QEEEEEETETNFPEPPQDQESSPIENDSSP

SEQ ID NO: 112 - protein, 31 aa, Homo sapiens
IQEEEEEETETNFPEPPQDQESSPIENDSSP

SEQ ID NO: 113 - protein, 32 aa, Homo sapiens
PIQEEEEEETETNFPEPPQDQESSPIENDSSP

SEQ ID NO: 114 - protein, 33 aa, Homo sapiens
IPIQEEEEEETETNFPEPPQDQESSPIENDSSP

SEQ ID NO: 115 - protein, 34 aa, Homo sapiens
IIPIQEEEEEETETNFPEPPQDQESSPIENDSSP

SEQ ID NO: 116 - protein, 35 aa, Homo sapiens
EIIPIQEEEEEETETNFPEPPQDQESSPIENDSSP

SEQ ID NO: 117 - protein, 36 aa, Homo sapiens
IEIIPIQEEEEEETETNFPEPPQDQESSPIENDSSP

SEQ ID NO: 118 - protein, 37 aa, Homo sapiens
DIEIIPIQEEEEEETETNFPEPPQDQESSPIENDSSP

SEQ ID NO: 119 - protein, 38 aa, Homo sapiens
EDIEIIPIQEEEEEETETNFPEPPQDQESSPIENDSSP

SEQ ID NO: 120 - protein, 39 aa, Homo sapiens
EEDIEIIPIQEEEEEETETNFPEPPQDQESSPIENDSSP

SEQ ID NO: 121 - protein, 40 aa, Homo sapiens
NEEDIEIIPIQEEEEEETETNFPEPPQDQESSPIENDSSP

SEQ ID NO: 122 - protein, 41 aa, Homo sapiens
KNEEDIEIIPIQEEEEEETETNFPEPPQDQESSPIENDSSP

SEQ ID NO: 123 - protein, 42 aa, Homo sapiens
PKNEEDIEIIPIQEEEEEETETNFPEPPQDQESSPIENDSSP

SEQ ID NO: 124 - protein, 43 aa, Homo sapiens
QPKNEEDIEIIPIQEEEEEETETNFPEPPQDQESSPIENDSSP

SEQ ID NO: 125 - protein, 44 aa, Homo sapiens
SQPKNEEDIEIIPIQEEEEEETETNFPEPPQDQESSPIENDSSP

SEQ ID NO: 126 - protein, 45 aa, Homo sapiens
SSQPKNEEDIEIIPIQEEEEEETETNFPEPPQDQESSPIENDSSP

SEQ ID NO: 127 - protein, 46 aa, Homo sapiens
TSSQPKNEEDIEIIPIQEEEEEETETNFPEPPQDQESSPIENDSSP

SEQ ID NO: 128 - protein, 47 aa, Homo sapiens
ETSSQPKNEEDIEIIPIQEEEEEETETNFPEPPQDQESSPIENDSSP

SEQ ID NO: 129 - protein, 48 aa, Homo sapiens
TETSSQPKNEEDIEIIPIQEEEEEETETNFPEPPQDQESSPIENDSSP

SEQ ID NO: 130 - protein, 49 aa, Homo sapiens
LTETSSQPKNEEDIEIIPIQEEEEEETETNFPEPPQDQESSPIENDSSP

SEQ ID NO: 131 - protein, 50 aa, Homo sapiens
GLTETSSQPKNEEDIEIIPIQEEEEEETETNFPEPPQDQESSPIENDSSP

SEQ ID NO: 132 - protein, 51 aa, Homo sapiens
VGLTETSSQPKNEEDIEIIPIQEEEEEETETNFPEPPQDQESSPIENDSSP

SEQ ID NO: 133 - protein, 52 aa, Homo sapiens [sequence omitted]

SEQ ID NO: 134 - protein, 53 aa, Homo sapiens
EVVGLTETSSQPKNEEDIEIIPIQEEEEEETETNFPEPPQDQESSPIENDSSP

SEQ ID NO: 135 - protein, 54 aa, Homo sapiens
EEVVGLTETSSQPKNEEDIEIIPIQEEEEEETETNFPEPPQDQESSPIENDSSP

SEQ ID NO: 136 - protein, 55 aa, Homo sapiens
KEEVVGLTETSSQPKNEEDIEIIPIQEEEEEETETNFPEPPQDQESSPIENDSSP

SEQ ID NO: 137 - protein, 56 aa, Homo sapiens
IKEEVVGLTETSSQPKNEEDIEIIPIQEEEEEETETNFPEPPQDQESSPIENDSSP

SEQ ID NO: 138 - protein, 57 aa, Homo sapiens
EIKEEVVGLTETSSQPKNEEDIEIIPIQEEEEEETETNFPEPPQDQESSPIENDSSP

SEQ ID NO: 139 - protein, 58 aa, Homo sapiens
IEIKEEVVGLTETSSQPKNEEDIEIIPIQEEEEEETETNFPEPPQDQESSPIENDSSP

SEQ ID NO: 140 - protein, 59 aa, Homo sapiens
TIEIKEEVVGLTETSSQPKNEEDIEIIPIQEEEEEETETNFPEPPQDQESSPIENDSSP

SEQ ID NO: 141 - protein, 60 aa, Homo sapiens
QTIEIKEEVVGLTETSSQPKNEEDIEIIPIQEEEEEETETNFPEPPQDQESSPIENDSSP

SEQ ID NO: 142 - protein, 61 aa, Homo sapiens
EQTIEIKEEVVGLTETSSQPKNEEDIEIIPIQEEEEEETETNFPEPPQDQESSPIENDSSP

SEQ ID NO: 143 - protein, 62 aa, Homo sapiens
KEQTIEIKEEVVGLTETSSQPKNEEDIEIIPIQEEEEEETETNFPEPPQDQESSPIENDSSP

SEQ ID NO: 144 - protein, 63 aa, Homo sapiens
KKEQTIEIKEEVVGLTETSSQPKNEEDIEIIPIQEEEEEETETNFPEPPQDQESSPIENDSSP

SEQ ID NO: 145 - protein, 64 aa, Homo sapiens
EKKEQTIEIKEEVVGLTETSSQPKNEEDIEIIPIQEEEEEETETNFPEPPQDQESSPIENDSSP

SEQ ID NO: 146 - protein, 65 aa, Homo sapiens
EEKKEQTIEIKEEVVGLTETSSQPKNEEDIEIIPIQEEEEEETETNFPEPPQDQESSPIENDSSP

SEQ ID NO: 147 - protein, 66 aa, Homo sapiens
AEEKKEQTIEIKEEVVGLTETSSQPKNEEDIEIIPIQEEEEEETETNFPEPPQDQESSPIENDSSP

SEQ ID NO: 148 - protein, 67 aa, Homo sapiens
SAEEKKEQTIEIKEEVVGLTETSSQPKNEEDIEIIPIQEEEEEETETNFPEPPQDQESSPIENDSSP

SEQ ID NO: 149 - protein, 68 aa, Homo sapiens
LSAEEKKEQTIEIKEEVVGLTETSSQPKNEEDIEIIPIQEEEEEETETNFPEPPQDQESSPIENDSSP

SEQ ID NO: 150 - protein, 69 aa, Homo sapiens
LLSAEEKKEQTIEIKEEVVGLTETSSQPKNEEDIEIIPIQEEEEEETETNFPEPPQDQESSPIENDSSP

SEQ ID NO: 151 - protein, 70 aa, Homo sapiens
VLLSAEEKKEQTIEIKEEVVGLTETSSQPKNEEDIEIIPIQEEEEEETETNFPEPPQDQESSPIENDSSP

SEQ ID NO: 152 - protein, 71 aa, Homo sapiens
IVLLSAEEKKEQTIEIKEEVVGLTETSSQPKNEEDIEIIPIQEEEEEETETNFPEPPQDQESSPIENDSSP

SEQ ID NO: 153 - protein, 72 aa, Homo sapiens
NIVLLSAEEKKEQTIEIKEEVVGLTETSSQPKNEEDIEIIPIQEEEEEETETNFPEPPQDQESSPIENDSSP

SEQ ID NO: 154 - protein, 73 aa, Homo sapiens
SNIVLLSAEEKKEQTIEIKEEVVGLTETSSQPKNEEDIEIIPIQEEEEEETETNFPEPPQDQESSPIENDSSP

SEQ ID NO: 155 - protein, 74 aa, Homo sapiens
KSNIVLLSAEEKKEQTIEIKEEVVGLTETSSQPKNEEDIEIIPIQEEEEEETETNFPEPPQDQESSPIENDSSP

SEQ ID NO: 156 - protein, 75 aa, Homo sapiens
PKSNIVLLSAEEKKEQTIEIKEEVVGLTETSSQPKNEEDIEIIPIQEEEEEETETNFPEPPQDQESSPIENDSSP

SEQ ID NO: 157 - protein, 76 aa, Homo sapiens
RPKSNIVLLSAEEKKEQTIEIKEEVVGLTETSSQPKNEEDIEIIPIQEEEEEETETNFPEPPQDQESSPIENDSSP

SEQ ID NO: 158 - protein, 77 aa, Homo sapiens
SRPKSNIVLLSAEEKKEQTIEIKEEVVGLTETSSQPKNEEDIEIIPIQEEEEEETETNFPEPPQDQESSPIENDSSP

SEQ ID NO: 159 - protein, 78 aa, Homo sapiens
CSRPKSNIVLLSAEEKKEQTIEIKEEVVGLTETSSQPKNEEDIEIIPIQEEEEEETETNFPEPPQDQESSPIENDSSP

SEQ ID NO: 160 - protein, 79 aa, Homo sapiens
TCSRPKSNIVLLSAEEKKEQTIEIKEEVVGLTETSSQPKNEEDIEIIPIQEEEEEETETNFPEPPQDQESSPIENDSSP

SEQ ID NO: 161 - protein, 80 aa, Homo sapiens
RTCSRPKSNIVLLSAEEKKEQTIEIKEEVVGLTETSSQPKNEEDIEIIPIQEEEEEETETNFPEPPQDQESSPIENDSSP

SEQ ID NO: 162 - protein, 81 aa, Homo sapiens
KRTCSRPKSNIVLLSAEEKKEQTIEIKEEVVGLTETSSQPKNEEDIEIIPIQEEEEEETETNFPEPPQDQESSPIENDSSP

SEQ ID NO: 163 - protein, 82 aa, Homo sapiens
WKRTCSRPKSNIVLLSAEEKKEQTIEIKEEVVGLTETSSQPKNEEDIEIIPIQEEEEEETETNFPEPPQDQESSPIENDSSP

SEQ ID NO: 164 - protein, 83 aa, Homo sapiens
EWKRTCSRPKSNIVLLSAEEKKEQTIEIKEEVVGLTETSSQPKNEEDIEIIPIQEEEEEETETNFPEPPQDQESSPIENDSSP

SEQ ID NO: 165 - protein, 84 aa, Homo sapiens
NEWKRTCSRPKSNIVLLSAEEKKEQTIEIKEEVVGLTETSSQPKNEEDIEIIPIQEEEEEETETNFPEPPQDQESSPIENDSSP

SEQ ID NO: 166 - protein, 85 aa, Homo sapiens
ENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 167 - protein, 86 aa, Homo sapiens
VENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 168 - protein, 87 aa, Homo sapiens
IVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 169 - protein, 88 aa, Homo sapiens; identical to SEQ ID NO: 12